(19) United States
(12) Patent Application Publication
(10) Pub. No.: US 2004/0136975 A1
(43) Pub. Date: Jul. 15, 2004

(54) Anthrax lethal factor inhibits tumor growth and angiogenesis

(76) Inventors: Nicholas S Duesbery, Grand Rapids, MI (US); Craig P Webb, Grand Rapids, MI (US); George F Vande Woude, Ada, MI (US)

Correspondence Address: VENABLE, BAETJER, HOWARD AND CIVILETTI, LLP, P.O. BOX 34385, WASHINGTON, DC, 20043-9998, US

(21) Appl. No.: 10/472,396

(22) PCT Filed: Mar. 22, 2002
(86) PCT No.: PCT/US02/08656

(51) Int. Cl. A61K 38/48

(52) U.S. Cl. 424/94.63

(57) ABSTRACT

A method for inhibiting cell angiogenesis comprises contacting cells associated with undesired angiogenesis with an effective amount of an inhibitor of MEK or of an enzyme that is a member of the MAPK family. MEK inhibitors include MEK-directed proteases such as Bacillus anthracis lethal factor or a functional derivative thereof. Organic small molecule inhibitors of MEK include PD98059, U0126 and PD184352. The above contacting may be performed in vivo, in a human or other mammalian subject. Also included is a method to treat a mammalian subject having a disease or condition associated with undesired angiogenesis or neovascularization, comprising administering to the subject an effective amount of a pharmaceutical composition that comprises an inhibitor of MEK or of an enzyme that is a member of the MAPK family, as noted above, and pharmaceutically acceptable carrier or excipient. The treatment method is useful for a disease or condition such as tumor growth, tumor invasion or tumor metastasis, wherein the angiogenesis inhibition results in reduction in size or growth rate of the tumor or its destruction.

### BACKGROUND OF THE INVENTION

1. Field of The Invention

The present invention in the fields of molecular/cell biology and medicine is directed to the use of anthrax Lethal Factor (LF), for example, in the form of anthrax lethal toxin (LeTx), as an agent for inhibition of angiogenesis and treatment of diseases and conditions associated with undesired angiogenesis, growth of primary and metastatic tumors.

2. Description of the Background Art

MEK's and MAPK Pathways

Mitogen activated protein kinase kinases (MAPKKs or MEKs) play pivotal roles in a variety of signal transduction pathways, aspects of which are critical for cell cycle progression and differentiation (Lewis, TS et al., (1998) in Adv. Canc. Res., eds., Vande Woude, GF et al., (Academic Press, San Diego) pp. 49-139). Mitogen activated protein kinases (MAPKs), their upstream activators and their downstream effectors define multiple, conserved, eukaryotic signal transduction pathways. Each of these pathways operates in parallel to the others, responding to particular extracellular stimuli both at a translational and post-translational level.

Thus, MEKs are upstream activators of members of the MAPK family. These members comprise extracellular-signal-regulated kinases (ERKs) also known as mitogen-activated protein kinases (MAPKs), for example, ERK 1 or ERK 2 which are the same as MAPK 1 or MAPK 2). Seven different MEK enzymes have been described. MEKs 1 and 2 phosphorylate and activate ERK 1 and 2 (=MAPK 1 and 2) in response to activation by the ras pathway. MEKs 1 and 2 are stimulated by mitogens or growth factors. MEKs 3, 4, and 6 have been implicated in regulation of the MAPK family member p38 MAPK (Derijard B et al., Science, 1995, 267:682-685; Raingeaud J et al., Mol Cell Biol, 1996, 16:1247-1255) which mediates cellular response to stimuli such as osmotic shock or cytokines (Lee J C et al., Nature, 1994, 372:739-746; Han J et al., Science, 1994, 265:808-811; Freshney N W et al., Cell, 1994, 78:1039-1049; Rouse J et al., Cell, 1994, 78:1027-1037) and also may play a role in monitoring spindle assembly during mitosis (Takenaka K et al., Science, 1998, 280:599-602). MEKs 4 and 7 regulate the activity of the MAPK family member known as “stress activated protein/jun kinase” (SAPK, JNK or SAPK/JNK), which in turn regulates the transcription factor jun (Derijard et al., supra). MEK 5 regulates the activity of ERK 5 (Zhou G et al., J Biol Chem, 1995, 270:12665-12669) and is activated in response to oxidative stress, hyperosmolarity and serum treatment (Kato Y et al., Nature, 1998, 395:713-716; Kato Y et al., EMBO J, 1997, 16:7054-7066).

Progression through the cell cycle requires the coordinated activities of the enzymatic cyclin-dependent kinases (cdk's) and their regulatory partners, the cyclins. Extracellular signals mediate cell cycle progression through G₁ in part through their regulation of D type cyclins and their partner cdk4/6. Importantly, sustained activation of ERK by extracellular growth factors is required for expression of cyclin D; expression of dominant negative MEK or ERK inhibits cyclin D expression (Lavoie J N et al., J Biol Chem, 1996, 271:20608-16; Albanese C et al., J Biol Chem, 1995, 270 :23589-97) and constitutively active MEK increases expression of cyclin D mRNA and protein (Cheng M et al., Proc Natl Acad Sci USA, 1998, 95:1091-6; Sewing A et al., Mol Cell Biol, 1997, 17:5588-97; Woods D et al., Mol. Cell Biol., 1997, 17:5598-5611). Consequently, mitogen-induced entry into S-phase of the cell cycle is blocked by antisense ERK mRNA (Pages G et al., Proc Natl Acad Sci USA, 1993, 90:8319-23) dominant negative ERK mutants (Troppmair J et al., J Biol Chem, 1994, 269:7030-5; Frost J A et al., Proc Natl Acad Sci USA, 1994, 91:3844-8), and small molecule inhibitors of MEK1/2 such as PD98059 (Dudley D T et al., Proc Natl Acad Sci USA, 1995, 92:7686-7689) or PD184352 (Sebolt-Leopold J S et al., Nat Med, 1999, 5:810-6)

MEKs also play a role in programmed cell death (reviewed in Raff M, Nature, 1998, 396:119-22). For example, survival of differentiated rat PC-12 pheochromocytoma cells in culture is dependent upon the presence of nerve growth factor (NGF) and removal of NGF from the medium causes an increase in the activities of p38 MAPK and JNK which is necessary and sufficient to induce apoptosis (Xia Z et al., Science, 1995, 270:1326-31). Interestingly, a decrease in ERK activity accompanies NGF withdrawal and expression of constitutively activated MEK1 prevents apoptosis induced by NGF withdrawal. These results indicate that apoptosis in NGF-differentiated PC-12 cells is regulated by opposing activities of ERK and p38 MAPK/JNK. Similar results have been obtained for paclitaxel-mediated apoptosis of transformed cells (MacKeigan J P et al., J Biol Chem, 2000, 275:38953-38956).

MEKs and Cancer

MEKs regulate cellular responses to mitogens as well as environmental stress. Inappropriate activation of these kinases contributes to tumorigenesis. Activated MAPK or elevated MAPK expression has been detected in a variety of human tumors including breast carcinoma and glioblastoma, as well as primary tumor cells derived from kidney, colon, and lung tissues (Hoshino, R. et al. (1999). Oncogene 18, 813-22; Salh, B et al. (1999) Anticancer Res 19, 741-8; Sivaraman, V S et al., (1997) J Clin Invest 99, 1478-83; Mandell, J W et al., 1998) Am J Pathol 153, 1411-23); Mansour, S J et al., (1994) Science 265, 966-970).

MEK-ERK signalling has also been shown to play a critical role in tumor metastasis (Jeffers M et al., Proc Natl Acad Sci USA, 1998, 95:14417-22; Ward Y et al., Mol Cell Biol, 2001, 21:5958-69; Webb C P et al., Proc Natl Acad Sci USA, 1998, 95:8773-8778) and may also be involved in tumor angiogenesis (Berra E et al., Cancer Metastasis Rev, 2000, 19:139-45; Dong G et al., Cancer Res, 2001, 61:5911-8; Giroux S et al., Cancer Res, 1999, 9:369-72).

Other MEK related pathways may also play a role in tumorigenesis. Activation of p38 MAPK is required for expression of and cellular response to vascular endothelial growth factor (VEGF) which promotes angiogenesis (Clauss M et al., Blood, 2001, 97:1321-9; Rousseau S et al., Oncogene, 1997, 15:2169-77; Sodhi A et al., Cancer Res, 2000, 60:4873-80).

Anthrax Lethal Factor

Anthrax lethal factor (LF), the principal virulence factor of anthrax toxin, has been demonstrated to selectively inactivate MEKs. LF is a protease that cleaves members of the MEK family including MEKs 1, 2 (Duesbery, N S. et al. (1998) Science 280:734-7; Vitale, G et al., (1998) Biochem Biophys Res Commun 248:706-11 and MEK3 (Pellizzari, R et al., (1999) FEBS Lett 462:199-204). LF-induced proteolysis of MEK1 blocks MAPK activation (Duesbery et al., supra; Colanzi, A et al., (2000) J Cell Biol 149:331-9). LF is produced by Bacillus anthracis, the Gram-positive bacterium responsible for the disease anthrax. B. anthracis produces an exotoxin consisting of three proteins; protective antigen (PA), LF, and edema factor (EF) (Duesbery, N S et al. (1999) Cell Mol Life Sci 55:1599-609). By itself PA is non-toxic (Thorne, C B et al. (1960). J. Bacteriol 79:450-455; Stanley, J. L. et al. (1960) J. Gen. Microbiol. 22:206-218). Rather, it serves to translocate EF and LF from the exterior to the cytoplasm of the host cell via the endosomal pathway. EF is an adenylyl cyclase enzyme that dramatically elevates intracellular cAMP concentrations (Leppla, S H (1982) Proc Natl Acad Sci USA 79:3162-6).

Combinations of EF plus PA (termed “edema toxin” of “EdTx”) cause skin edema characteristic of anthrax but are not toxic when injected intravenously into mice or rats. By contrast, combinations of LF plus PA (=lethal toxin or LeTx) do not cause skin edema but are lethal when injected intravenously (Stanley, J L et al. (1961) J Gen. Microbiol. 26:49-66; Beall, F A et al. (1962) J. Bacteriol. 83:1274-1280).

Angiogenesis

Angiogenesis, the formation of new capillaries from pre-existing ones (Folkman, J, N. Engl. J. Med., 1971, 285:1182-1186; Hanahan D. et al., Cell, 1996, 86:353-364), is a normal part of embryonic development, wound healing and female reproductive function. However, angiogenesis also plays a pathogenic role in the establishment and progression of certain diseases. Cancer, rheumatoid arthritis and diabetic retinopathy are examples of such diseases (Carmeliet P. et al., Nature, 2000, 407:249-257). Anti-angiogenic therapy holds promise in inhibiting the progression of these diseases.

Angiogenesis can be triggered by several pro-angiogenic cytokines. In the setting of cancer, tumor cells under hypoxic conditions secrete VEGF and/or fibroblast growth factor (bFGF). These proteins diffuse and bind to specific receptors on endothelial cells (ECs) in the local vasculature, perturbing the balance of pro- and anti-angiogenic forces in favor of angiogenesis. As a consequence of binding these proteins, ECs are activated to (a) secrete enzymes that induce remodeling of the associated tissue matrix, and (b) change the patterns and levels of expression of adhesion molecules such as integrins. Following matrix degradation, ECs proliferate and migrate toward the hypoxic tumor, resulting in the generation and maturation of new blood vessels.

Interestingly, many anti-angiogenic factors result from the degradation of matrix proteins—i.e., are a result of the action of pro-angiogenic enzymes. Examples include endostatin, a fragment of collagen XIII (O'Reilly, M. S. et al., Cell 1997, 88:277-285); kringle 5 of plasminogen (O'Reilly, M. S. et al., Cell, 1994, 79:315-328) and PEX, the C-terminus non-catalytic subunit of MMP-2 (Brooks P. C. et al., Cell, 1998, 92:391-400).

The concept has emerged that, due to the abundance of pro-angiogenic-factors, these anti-angiogenic molecules are unable to overcome the pro-angiogenic balance in a primary tumor. However, since they are secreted into circulation, these anti-angiogenic molecules are capable of inhibiting angiogenesis at other locations where tumor cells may have begun to invade. Consequently, micro-metastases comprising these tumor cells at these new locations remain dormant. This hypothesis explains the puzzling observation made by surgeons many years ago: at various times after surgical removal of a primary tumor in a patient with no obvious metastatic disease, the patient returns with advanced metastatic disease.

Thus, clinical intervention by treatment with one or more of the anti-angiogenic agents could inhibit the angiogenic process and halt tumor growth as well as metastasis. Significant evidence in the literature (cited above) supports this notion.

Unregulated angiogenesis contributes to the pathology of not only many neoplastic diseases but also a number of non-neoplastic diseases associated with abnormal neovascularization including arthritis, various ocular disorders, and psoriasis. See, for example, Moses et al., 1991, Biotech. 9: 630-634; Folkman et al., 1995, N. Engl. J. Med., 333:1757-1763; Auerbach, R et al., 1985, J. Microvasc. Res. 29:401-411; Folkman, 1985, Adv Canc Res 43:175-203; Patz, A, 1982, Am. J. Opthalmol. 94:715-743; Patz, A, 1982, Am. J. Opthalmol. 94:552-554. Maintenance of the avascularity of the cornea, lens, and trabecular meshwork is crucial for vision as well as to normal ocular physiology. A number of ocular diseases, some of which lead to blindness, result from ocular neovascularization and include diabetic retinopathy, neovascular glaucoma, ocular inflammatory diseases and ocular tumors (e.g., retinoblastoma). Other eye diseases which are associated with neovascularization, including retrolental fibroplasia, uveitis, retinopathy of prematurity, and macular degeneration. About twenty eye diseases are associated with choroidal neovascularization and about forty with iris neovascularization (Waltman D D et al., 1978, Am. J. Ophthal. 85:704-710 and Gartner, S. et al., 1978, Surv. Ophthal. 22:291-312. Current treatments of these diseases, especially once neovascularization has occurred, are frequently inadequate to stave off blindness. Studies have suggested that vaso-inhibitory factors which are present in normal ocular tissue (cornea and vitreous) are lost in the diseased states.

Thus, while inactivation of MEKs underlies the pathogenesis of anthrax, their inappropriate activation contributes to the pathogenesis of cancer. This gave rise to the present inventors' conception of the present invention of using anthrax LeTx and other MEK inhibitors as antiangiogenic agents, as well serving as the basis of related inventions by the present inventors and/or their colleagues for the treatment of cancer (Duesbery et al., WO 99/50439) including, in particular, human melanoma (Koo et al., WO02/17952).

### SUMMARY OF THE INVENTION

Anthrax lethal factor (“LF”) is a protease, one component of Bacillus anthracis exotoxin, which cleaves many of the MEKs. Given the importance of MEK signaling in tumorigenesis, the present inventors assessed the effects of anthrax lethal toxin (LeTx) upon tumor cells. LeTx effectively inhibited MAPK activation in V12H-ras transformed NIH 3T3 cells. In vitro, treatment of transformed cells with LeTx caused them to revert to a non-transformed morphology, inhibited their ability to form colonies in soft agar and to invade Matrigel, without markedly affecting cell proliferation. In vivo, LeTx inhibited growth of ras-transformed cells implanted in athymic nude mice, in some cases causing tumor regression, at concentrations that caused no apparent systemic toxicity.

Unexpectedly, LeTx also greatly decreased tumor neo-vascularization. The present inventors therefore conceived that LeTx, though the LF component, and other proteins or small molecules with similar modes of biochemical action, are potent inhibitors of angiogenesis, including tumor angiogenesis.

The present invention is directed specifically to a method for inhibiting cell migration, cell invasion, cell proliferation or angiogenesis, or for inducing apoptosis, comprising contacting cells associated with undesired cell migration, invasion, proliferation or angiogenesis with an effective amount of an inhibitor of MEK or of an enzyme that is a member of the MAPK family. Preferably, the method is for inhibiting angiogenesis.

In the above method, the inhibitor of MEK is preferably a MEK-directed protease, such as Bacillus anthracis lethal factor or a functional derivative or homologue thereof.

In another embodiment, the MEK inhibitor is an organic small molecule, preferably PD98059, U0126 or PD184352.

In the above method, the inhibitor may be an inhibitor of a MAPK family member selected from the group consisting of ERK 1, ERK2, p38 kinase and JNK. A preferred embodiment targets p38 kinase, where a preferred inhibitor is SB203580.

The above method may be performed so that the contacting is in vivo, such as the contacting in a mammalian subject, preferably a human. In one embodiment, the subject has a tumor and the inhibition of angiogenesis results in cessation of growth or a measurable regression of a primary or metastatic tumor.

This invention also provides a method for inhibiting angiogenesis in a mammalian subject, preferably a human, comprising administering to a mammalian subject in need of such inhibition an angiogenesis-inhibiting amount of a pharmaceutical composition that comprises:

(a) an inhibitor of MEK or of an enzyme that is a member of the MAPK family; and

(b) a pharmaceutically acceptable carrier or excipient, thereby inhibiting the angiogenesis.

The invention includes a method for treating a mammalian subject, preferably a human, having a disease or condition associated with undesired cell migration, invasion, proliferation, or angiogenesis, comprising administering to the subject an effective amount of a pharmaceutical composition that comprises:

(a) an inhibitor of MEK or of an enzyme that is a member of the MAPK family; and

(b) a pharmaceutically acceptable carrier or excipient, thereby treating the subject.

In a preferred embodiment of the above method, the disease or condition is associated with undesired angiogenesis or neovascularization

As above, in this treatment method the inhibitor is preferably a MEK inhibitor such as a MEK-directed protease, preferably Bacillus anthracis lethal factor or a functional derivative or homologue thereof.

In another embodiment of the treatment method, the inhibitor is an organic small molecule; preferred MEK inhibitors of this class are PD98059, U0126 and PD184352.

The treatment method may employ an inhibitor of a MAPK family member selected from the group consisting of ERK 1, ERK2, p38 kinase and JNK. A preferred target is p38 kinase, using, for example, the inhibitor is SB203580.

Diseases or conditions that are treated by the above method are tumor growth, tumor invasion or tumor metastasis wherein the angiogenesis inhibition results in reduction in size or growth rate of the tumor or destruction of the tumor. Preferred a targets are solid tumors including brain tumors.

### BRIEF DESCRIPTION OF THE DRAWINGS

FIGS. 1a-1h show the effects of LeTx upon MAPK activation and cell morphology. Immunoblotting of lysates from non-transformed (pDCR NIH 3T3) (FIG. 1a) and V12H-ras transformed NIH 3T3 cells (FIG. 1b) show loss of NH₂-terminal epitopes of MEK and phospho-epitopes of MAPK following treatment of cells with LeTx but not in control cells treated with medium alone, 100 ng/ml PA plus inactive 10 ng/ml LF(E687C), PA plus LF (100 ng/ml PA plus 10 ng/ml LF), or PD98059 (50 μM from a 50 mM stock in DMSO). Non-transformed (FIG. 1c) cells possessed an irregular, flattened morphology which was not substantially altered by 24 hr. exposure to LeTx (FIG. 1d). In contrast, following 24 hr. LeTx treatment the well-defined, elongated, spindle-like shape of V12H-ras transformed NIH 3T3 cells (FIG. 1e), reverted to that resembling a non-transformed cell (FIG. 1f). Immunostaining of V12H-ras transformed NIH 3T3 cells incubated in the absence (FIG. 1g) or presence (FIG. 1) of LeTx for 24 hr. for actin (green) showed actin stress fibers formed following LeTx treatment.

FIGS. 2a-2e show the effects of LeTx upon anchorage independent colony formation and extracellular matrix invasion. We evaluated the ability of V12H-ras-transformed cells to form colonies in Noble agar (FIGS. 2a, 2b) or invade Matrigel (FIGS. 2c, 2d) in the presence (FIGS. 2b, 2d) or absence (FIGS. 2a, 2c) of LeTx. FIG. 2e shows results of assays of the levels of cathepsin L by immunoblotting lysates of V12H-ras transformed NIH 3T3 cells which had been treated with medium alone, PA plus inactive LF(E687C), or LeTx. Blots were stripped and reprobed with antibodies raised against β-tubulin to control for lane loading.

FIGS. 3a-3d show the effects of LeTx upon V12H-ras transformed NIH 3T3 xenografts in athymic nude mice. Growth of tumors derived from V12H-ras transformed NIH 3T3 cells was measured after either sham injection or injection with Hank's buffered saline solution (HBSS) (FIG. 3a) or after injection with either HBSS or HBSS containing PA and LF (FIG. 3b). Open symbols indicate the tumor on the left side, closed symbols the tumor on the right. Tumor size is expressed as the product of their measured length and width. Arrow heads on the x-axis indicate the times of injection. The appearance of tumors from Group A (FIG. 3c) and Group B (FIG. 3d) are shown adjacent to a ruler indicating tumor size (mm).

FIGS. 4a-4h are a series of photomicrographs showing a histological analyses of tumors derived from LeTx injected mice. Tumors excised from groups A (FIGS. 4a-4d) and B (FIGS. 4e-4h) mice were sectioned and immunostained with antibodies to the angiogenic markers CD31 (FIGS. 4a, 4e) or CD34 (FIGS. 4b, 4f) or stained by hematoxylin and eosin (H&E) and shown at low (40×) and high (100×) magnification.

### DESCRIPTION OF THE PREFERRED EMBODIMENTS

Based upon previous work demonstrating a prominent role for the MEK-MAPK signalling pathway in cancer (discussed above), the present inventors conceived that LeTx, thorough LF, is a potent inhibitor not only of ras-mediated oncogenic transformation and in vivo tumor growth per se but also tumor vascularization. The effects on vascularization are apparent at concentrations which have no apparent side effects. The inventors therefore evaluated the effect of LeTx upon (a) ras-mediated transformation iii vitro and in vivo, (b) and on tumor growth in vivo, and (c) on tumor vascularization.

Copending U.S. application Ser. No. 09/623,104 and PCT application US99/07126 published as WO 99/50439 (Duesbery et al., “Anthrax Lethal Factor is a MAPK Kinase Protease”) and U.S. application Ser. No. 09/942,940 and PCT/US01/27063, published as WO02/17952 (Koo et al., “Inhibition of Mitogen-Activated Protein Kinase(MAPK) Pathway as Selective Therapeutic Strategy Against Melanomas”) are incorporated by reference in entirety.

As it is used herein, the term LF is intended to include close LF homologues, functional derivatives and mimetics (whether or not the latter terms are listed after an occurrence of “LF”). These are described below. LF inhibits the MAPK pathway by proteolytically cleaving a MEK family member. This discovery also provides means for identifying novel therapeutic agents, including smaller organic molecules that acting as LF mimetics or modulators, that either cleave or promote proteolytic cleavage of a MEK, thereby inhibiting the MAPK signal transduction pathway. Such agents are useful for treating cancer.

By its action on MEKs, LF can reverse or attenuate numerous cellular changes associated with oncogenic transformation, including, but not limited to, characteristic cellular morphology, intracellular patterns of actin distribution, rates of proliferation and anchorage-independent growth. In addition, LF (or the PA component with which it is preferably administered) may be further modified to specifically target cancer cells in a more selective manner, thereby rendering it particularly useful as a cancer therapeutic.

Modulators that activate or promote LF proteolytic activity may be used along with LF, its homologues or mimetics to promote their anticancer activity.

Functional homologues and derivatives, mimetics or modulators of LF can be identified using various assays based on techniques described herein.

A “LF mimetic” is an agent, generally a polypeptide or peptide molecule, that recognizes a MEK as a substrate and cleaves the MEK at the same site cleaved by full-length, native LF. Thus, LF mimetics include homologues, peptides of LF, conservative substitution variants, as well as deletion variants that retain the protease active site and proteolytic action on MEKs. LF mimetics are tested using assays for LF activity, e.g., MEK mobility shift assays, MOS-induced activation of MAPK in oocytes and myelin basic protein (MBP) phosphorylation, as are known in the art. In assessing a LF mimetic, LF is generally the positive control for protease activity. Mimetic activity is at least about 20% of the activity of this control, more preferably between about 50-100% of the positive control.

“Modulators” of LF are agents that activate (promote, enhance, increase) or inhibit (suppress, block, decrease) LF proteolytic activity and are identified by assays in vitro or in vivo assays of this activity or downstream activities.

While the present disclosure is directed primarily to LF from Yersinia pestis or Yersinia pseudotuberculosis, it is to be understood that homologues of LF from other Yersinia species, and mutants thereof, that possess the characteristics disclosed herein are intended within the scope of this invention. Also included are “functional derivatives” of LF, which is means a variant, “fragment,” or “chemical derivative” of LF, which terms are defined below. A functional derivative retains at least a portion of the relevant LF activity, that of proteolysis of MEK1 (or another MEK) which permits its utility in accordance with the present invention.

As noted above, to date, at least seven different MEKs have been identified, which are numbered MEK 1-MEK 7. MEK 4 and MEK 7 activate JNK. MEK 3 and 8 activate p38 MAPK. According to this invention, inhibition of p38 MAPK or JNK results in inhibition of angiogenesis.

Migration of smooth muscle cells, which contribute to angiogenesis, in response to various growth factors and cytokines is blocked by SB203586, an inhibitor of p38 MAPK (Hedges J C et al., J Biol Chem 1999, 274:24211-24219). Activation of p38 MAPK results in phosphorylation of HSP27 (which may modulate F-actin polymerization) and inhibition of p38 MAPK inhibits this phosphorylation. Expression of activated mutant MAPK kinase 6b(E), an upstream activator for p38 MAPK, increased cell migration, whereas overexpression of a p38α MAPK dominant negative mutant and an HSP27 phosphorylation mutant blocked cell migration completely (Hedges et al., supra). Because activation of the p38 MAPK pathway by growth factors and proinflammatory cytokines regulates SMC migration, it may contribute to pathological states associated with angiogenesis. Thus, use of some of the kinase inhibitors described herein may be used to inhibit this particular component of angiogenesis.

The JNK enzyme and pathway is involved in EC motility (Shin E Y et al., Exp Mol Med, 2001, 33:276-83). Stable transfectant ECs expressing oncogenic H-Ras (Leu 61 showed enhanced angiogenic potential and motility compared to control EC cells. JNK was constitutively activated in these transfectants. Pretreatment with JNK-specific inhibitors, curcumin and trans-retinoic acid, decreased the basal motility and motility stimulated by JNK agonists (e.g., TNFα and anisomycin) of the transfected EC cells in a dose-dependent manner.

Angiogenesis promoted by TNFα is mediated, at least in part, by ephrin A1, a member of the ligand family for Eph receptor tyrosine kinases. Cheng N et al., J Biol Chem, 2001, 276:13771-7, showed that ephrin A1 induction was blocked by inhibition of p38 MAPK or SAPK/JNK, but not p42/44 MAPK, using either selective chemical inhibitors or dominant-negative forms of p38 MAPK or TNF receptor-associated factor 2. Thus TNF-α-induces ephrin A1 expression through JNK and p38 MAPK signaling pathways but not p42/44 MAPK.

Heregulin stimulates VEGF secretion from breast cancer cells that can result in increased EC migration. This action is inhibited by anti-VEGF-neutralizing antibody or SB 203580 (Xiong S et al., Cancer Res, 2001, 61:1727-32). Heregulin activates ERK, Akt kinase, and p38 MAPK; however, only the specific inhibitor of p38 MAPK (SB 203580), but not an ERK inhibitor PD98059 nor an inhibitor of phosphatidylinositol 3-kinase-Akt pathway (Wortmannin), blocks up-regulation of VEGF by heregulin. Thus, receptor mediated activation of p38 MAPK to enhance VEGF transcription via an upstream heregulin response element, leads to increased VEGF secretion by cancer cells and a subsequent angiogenic response.

Using targeted disruption of the p38α MAPK gene, Mudgett J S et al., Proc Natl Acad Sci USA 2000 97:10454-9 observed homozygous embryonic lethality because of severe defects in placental development. p38α mutant placentas displayed lack of vascularization of the labyrinth layer as well as increased rates of apoptosis, consistent with a defect in placental angiogenesis. p38α mutants also displayed abnormal angiogenesis in the embryo proper as well as in the visceral yolk sac. These requirements for p38α MAPK function in diploid trophoblast development and placental vascularization suggest a more general role for p38 MAPK signaling in embryonic angiogenesis.

The foregoing observations, and those discussed in the Background (Clauss et al.; Rousseau et al.; Sodhi A et al., supra) serve in part as a basis for the present inventors conception of the use of additional kinase inhibitors, primarily those acting on p38 MAPK or JNK, either alone or in conjunction with MEK inhibitors, as a therapeutic or prophylactic approach to inhibit angiogenesis.

A functional homologue (described in more detail in a separate section below) is required to possess the characteristic of having a proteolytic action on MEK1. In view of this functional characterizations, homologous proteins to LF from other bacterial species, even proteins not yet discovered, fall within the scope of the invention provided that these proteins have the recited biochemical and biological activity. It is within the skill in the art to obtain and express such a protein using DNA probes based on the sequence of LF or Salmonella or plant-derived homologues already characterized. Then, the protein's biochemical and biological activity can be tested readily using art-recognized methods such as those described herein, for example, a standard gel mobility shift assay for proteolysis of the substrate protein MEK1, or inhibition of MEK1-mediated phosphorylation of its natural substrate, MAPK, or of a model substrate.

A “variant” of LF refers to a molecule substantially identical to either the full protein or to a fragment thereof in which one or more amino acid residues have been replaced (substitution variant) or which has one or several residues deleted (deletion variant) or added (addition variant). A “fragment” of LF refers to any subset of the molecule, that is, a shorter polypeptide of the full length protein.

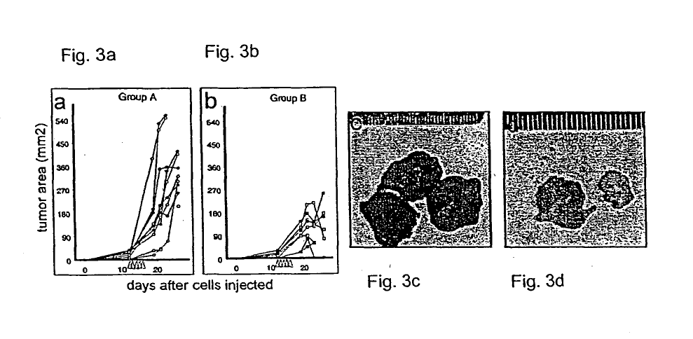

A preferred group of LF variants are those in which at least one amino acid residue and preferably, only one, has been substituted by different residue. For a detailed description of protein chemistry and structure, see Schulz, GE et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions which may be made in the protein molecule may be based on analysis of the frequencies of amino acid changes between a homologous protein of different species, such as those presented in Table 1-2 of Schulz et al. (supra) and FIGS. 3-9 of Creighton (supra). Based on such an analysis, conservative substitutions are defined herein as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, Trp.

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those which do not produce radical changes in the characteristics of the protein in terms of its proteolytic activity. However, when it is difficult to predict the exact effect of the substitution, deletion or insertion in advance of doing so, one skilled in the art will appreciate that the effect can be evaluated by routine screening assays such as those described here, without requiring undue experimentation.

Whereas shorter chain variants can be made by chemical synthesis, for the present invention, the preferred longer chain variants are typically made by site-specific mutagenesis of the nucleic acid encoding the polypeptide, expression of the variant nucleic acid in cell culture, and, optionally, purification of the polypeptide from the cell culture, for example, by immunoaffinity chromatography using specific antibody immobilized to a column (to absorb the variant by binding to at least one epitope).

The activity of a variant present in a cell lysate or a more highly purified preparation is screened in a suitable screening assay for the desired characteristic, preferably the proteolysis of a MEK. It is also possible to follow the immunological character of the protein molecule is assayed by alterations in binding to a given antibody, and may measured by competitive immunoassay. Biochemical or biological activity is screened in an appropriate assay, as described below.

A “detectable moiety” or label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens; and proteins for which antisera or monoclonal antibodies are available.

A protein is detectably labeled if it is bound, either covalently, through a linker, or through ionic, van der Waal's or hydrogen bonds, to a label such that the presence of the protein is detected by detecting the presence of the label.

Fusion Proteins

The present invention utilizes fusion proteins comprising LF (or its homologue or functional derivative) that are fused to another peptide or polypeptide that confers useful properties on the fusion protein such as stability.

PA is one of three protein components of the “lethal” or “anthrax” toxin produced by B. anthracis. The 83 kDa PA binds to a cell surface receptor present on almost all vertebrate cells, and its C-terminus is necessary for this binding (Singh, Y et al., J. Biol. Chem. 264:19103-19107 (1989); Novak, J. et al., J. Biol. Chem. 267:17186-17193 (1992)). After binding, PA is specifically cleaved by a protease (e.g., furin, clostripain or trypsin), releasing a 20 kDa N-terminal PA fragment while a 63 kDa C-terminal PA fragment (PA63) remains bound. PA63, also referred to as “processed PA,” contains the receptor binding site at its C-terminus. PA63 forms a heptameric membrane-inserted channel which mediates the entry of the two other protein components of the complex (LF and EF) into the cytosol via the endosomal pathway (Gordon et al., Infect. Immun. 56:1066-1069 (1988); Milne et al., J. Biol Chem. 269:20607-20612 (1994)).

The term “PA” refers a PA protein (or functional homologue or derivative) that has its receptor binding site intact and functional. U.S. Pat. Nos. 5,591,631 and 5,677,274 (incorporated by reference in their entirety) describe PA fusion proteins that target PA to particular cells, such as cancer cells and HIV-infected cells, using as fusion partners ligands for receptors on the targeted cells.

It is to be understood that use of LF and its homologues, as described herein, as antiangiogenic and/or antitumor agents, requires administration in conjunction with PA to achieve transport into cells.

Chemical Modification of the Protein

A “chemical derivative” of LF contains additional chemical moieties not normally a part of the protein. Covalent modifications of the protein are included within the scope of this invention. Such modifications may be introduced into the molecule by reacting targeted ammo acid residues with an organic derivatizing agent that is capable of reacting with selected side chains or terminal residues.

Such chemically modified and derivatized moieties may improve the protein's solubility, absorption, biological half life, and the like. These changes may eliminate or attenuate undesirable side effects of the protein in vivo. Moieties capable of mediating such effects are disclosed, for example, in Remington 's Pharmaceutical Sciences, Mack Publishing Company, Easton Pa. (Gennaro 18th ed. 1990).

Cysteinyl residues most commonly are reacted with α-haloacetates (and corresponding amines) to give carboxymethyl or carboxyarmdomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate (pH 5.5-7.0) which agent is relatively specific for the histidyl side chain. p-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents reverses the charge of the lysinyl residues. Other suitable reagents for derivatizing α-amino-containing residues include imidoesters such as methylpicolinimidate; pyridoxal phosphate; pyridoxal; chloroborohydride; trinitrobenzenesulfonic acid; O-methylisourea, 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKₐ of the guanidine functional group. Furthermore, these reagents may react with the groups of lysine as well as the arginine ε-amino group.

Modification of tyrosyl residues has permits introduction of spectral labels into a protein or peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide.

Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention.

Derivatization with bifunctional agents is useful for cross-linking the polypeptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other chemical modifications include hydroxylation of proline and lysine, phosphoiylation of the hydroxyl groups of seryl or threonyl residues, methylation of the α-amino groups of lysine, arginine and histidine side chains (T. E. Creighton, Proteins: Structure and Molecule Properties, W.H. Freeman & Co., San Francisco, pp. 79-86 (1983)), acetylation of the N-terminal amine, and, in some instances, amidation of the C-terminal carboxyl group.

Homologues

Homologues of LF (or peptide fragments or fusion proteins thereof) share sequence similarity with LF and also exhibit anti-angiogenic and anti-tumor activity. A functional homologue must possess the biochemical and biological activity, preferably MEK-inhibiting, ant-angiogenic and anti-tumor activity which can be tested using ill vitro or ill vivo methods described herein. In view of this functional characterization, use of homologous LF proteins from other species, including proteins not yet discovered, falls within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, or 90% of the length of the reference sequence. The amino acid residues (or nucleotides from the coding sequence) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

Thus, a homologue of the LF described herein is characterized as having (a) functional activity of native LF, and (b) sequence similarity to native LF when determined above, of at least about 30% (at the amino acid level), preferably at least about 50%, more preferably at least about 70%, even more preferably at least about 90%.

It is within the skill in the art to obtain and express such a protein using DNA probes based on the disclosed sequences of LF. Then, the protein's biochemical and biological activity can be tested readily using art-recognized methods such as those described herein. A biological assay in vitro or in vivo, as described herein will indicate whether the homologue has the requisite activity to qualify as a “functional” homologue.

Basic texts disclosing general methods of molecular biology, all of which are incorporated by reference, include: Sambrook, J. et al., Molecular Cloning: A Laboratory Manual, 2^{nd} Edition, Cold Spring Harbor Press, Cold Spring Harbor, N.Y., 1989; Ausubel, F. M. et al. Current Protocols in Molecular Biology, Vol. 2, Wiley-Interscience, New York, (current edition); Kriegler, Gene Transfer and Expression: A Laboratory Manual (1990); Glover, D. M., ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J. D. et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

The gene and nucleotide sequence encoding LF has been described. The gene encoding PA (assigned Genbank accession no. M22589) has been cloned and sequenced (Ivins, B E et al., Infect. Immun. 54:537-542 (1986); Welkos, S L et al., Gene 69:287-300 (1988); U.S. Pat. No. 5,591,631, U.S. Pat. No. 5,677,274; Leppla, S H, “Anthrax Toxins,” In: Handbook of Natural Toxins: Bacterial Toxins and Virulence Factors in Disease, Moss, J. et al., eds., Dekker, New York, 1995). The gene is encoded at the pag locus on the plasmid pXO1(formerly known as pBA1) (Mikeskell et al., Infect. Immun. 39:371-376 (1983)). The genes contains a 2319 bp-long open reading frame of which 2205 bp encode an A/T-rich (69%) cysteine-free, 735 amino acid (83 kDa) secreted protein. The protein has Swiss Prot accession number P13423. The genes encoding MEK1 and MEK-2 have been cloned and sequenced. MEK1 has been assigned GenBank accession no. L11284, and the accession no. for MEK-2 is L11285 (see, e.g., Zheng et al., J. Biol. Chem. 268:11435-11439 (1993)).

Peptidomimetics

A preferred type of LF-mimetic described herein is a peptidomimetic compound which mimics the biological effects of LF or of a biologically active peptide thereof. A peptidomimetic agent may be an unnatural peptide or a non-peptide agent that recreates the stereospatial properties of the binding elements of LF such that it has the binding activity or biological activity of LF. Similar to biologically active peptides, a peptidomimetic will have a binding face (which interacts with any ligand to which LF binds, such as a MEK enzyme) and a non-binding face. Again, similar to LF or its peptide, the non-binding face of a peptidomimetic will contain functional groups which can be modified, for example, by various therapeutic moieties without modifying the binding face of the peptidomimetic. A preferred embodiment of a peptidomimetic would contain an aniline on the non-binding face of the molecule. The NH₂-group of an aniline has a pKa˜4.5 and could therefore be modified by any NH₂-selective reagent without modifying any NH₂ functional groups on the binding face of the peptidomimetic. Other peptidomimetics may not have any NH₂ functional groups on their binding face and therefore, any NH₂, without regard for pKₐ could be displayed on the non-binding face as a site for conjugation. In addition other modifiable functional groups, such as —SH and —COOH could be incorporated into the non-binding face of a peptidomimetic as a site of conjugation. A therapeutic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptide nature.

Peptidomimetic compounds, either agonists, substrates or inhibitors, have been described for a number of bioactive peptides such as opioid peptides, VIP, thrombin, HIV protease, etc. Methods for designing and preparing peptidomimetic compounds are known in the art (Hruby, V J, Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that possess at least the binding capacity and specificity of LF and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the cystallographically-derived three-dimensional structure of LF or an active peptide of LF either free or bound in complex with a ligand such as a MEK or an active site mimic of thr MEK protein. Alternatively, the structure of a LF peptide bound to its ligand can be gained by the techniques of nuclear magnetic resonance spectroscopy. The better knowledge of the stereochemistry of the interaction of the peptide with its ligand will permit the rational design of useful peptidomimetic agents for inactivating MEK and inhibiting angiogenesis. The structure of LF or an LF in the absence of ligand could also provide a scaffold for the design of mimetic molecules.

Small Molecule Inhibitors of MEK

Also intended within the scope of this invention are treatment methods that utilize a combination of LF or an LF homologue with one or more small organic molecule inhibitors of MEK (or inhibitors of the MAPK pathway acting at different steps). As used herein, “small molecules” are organic chemical entities that are not biological macromolecules such as proteins or peptides. The small molecule inhibitors generally have a molecular mass of less than about 2000 Da, preferably less than about 1000 Da, more preferably less than about 500 Da. In a preferred embodiment, inhibition of MEK by the small molecule inhibitor PD98059 results in the efficient antiangiogenic and antitumor effects.

Other small molecule inhibitors of the MAPK pathway or related pathways useful in the present invention include the MEK inhibitors PD184352 (Parke-Davis) (Sebolt-Leopold et al., supra) and U0126 (DuPont) (Favata, M et al., J. Biol. Chem. 273:18623-18632 (1998)), the p38 MAPK inhibitor SB203580 (Schering-Plough) (Cuenda, A et al., FEBS Lett. 364:229-233 (1995)), and the like. Thee MEK inhibitors of this group are known to be, or are expected to be, cytotoxic to certain tumor cells.

The chemical structures of the above small molecule inhibitors are shown below:

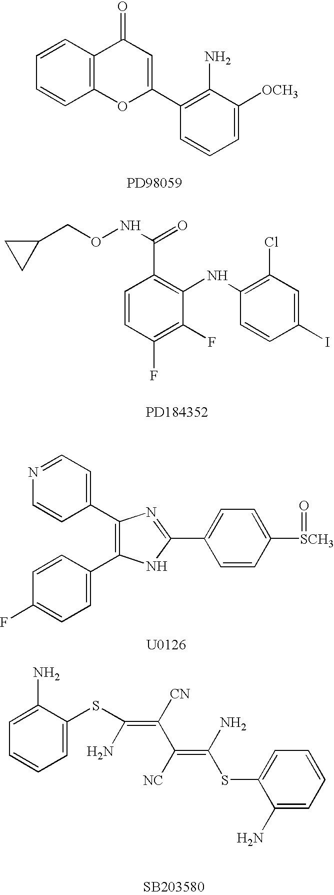

Assays to Identify Agents with MEK-Inhibitory and Antiangiogenic/Antitumor Activity or Modulators of Such Activities

Assays based on cell proliferation and tumor suppression are useful to detect MEK inhibitors such as LF functional derivatives or modulators, which are useful in inhibiting abnormal cellular proliferation and transformation, leading to antiangiogenic effects. Where the assays below are discussed in terms of LF or derivative, they are applicable for the evaluation of any candidate inhibitor.

In Vitro Testing of Compositions

A. Binding to Immobilized MEK in a 96 Well Plate

BINDING to immobilized MEK is carried out either by a competition assay with a known ligand, such as biotin-LF, or by direct binding of the compound being tested when the compound is labeled, e.g., biotinylated. Plates are coated at room temperature with MEK in Tris buffer-saline (TBS) (200 ng/well). After incubation for 2 hours, wells are washed with TBS, then 1% BSA/TBS/Tween-20 is added to each well and incubate at 37° C. for two hours. LF that had been previously biotinylated (e.g., with EZ-Link from Pierce Chemicals according to the manufacturers instructions), is added to the plate at a concentration of 10 nM and appropriate concentrations of the test compound as competitor. The plates are incubated at room temperature and then washed with TBS/Tween-20. Avidin-HRP is added, incubated for 20 minutes at room temperature, washed with TBS/Tween-20 and the chromogenic substrate is added. The reaction is stopped with sulfuric acid and the plate read at 490 nm.

B. Assay for EC Migration

For EC migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as HUVEC, which have been detached from monolayer culture using trypsin, are diluted to a final concentration of about 10⁶ cells/mL with serum-free media and 0.2 mL of this cell suspension is added to the upper chamber of each transwell. Inhibitors to be tested are added to both the upper and lower chambers, and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated.

C. Biological Assay of Anti-Invasive Activity

The compositions of the invention are tested for their anti-invasive capacity. The ability of cells such as ECs or tumor cells (e.g., PC-3 human prostatic carcinoma) cells to invade through a reconstituted basement membrane (Matrigel®) in an assay known as a “Matrigel® invasion assay” as described in detail by Kleinman et al., Biochemistry 25: 312-318,1986 and Parish et al., Int. J. Cancer 52:378-383,1992. Matrigel® is a reconstituted basement membrane containing type IV collagen, laminin, heparan sulfate proteoglycans such as perlecan, which bind to and localize bFGF, vitronectin as well as transforming growth factor-β (TGFβ), urokinase-type plasminogen activator (uPA), tissue plasminogen activator (tPA), and the serpin known as plasminogen activator inhibitor type 1 (PAI-1) (Chambers et al., Canc. Res. 55:1578-1585, 1995). It is accepted in the art that results obtained in this assay for compounds which target extracellular receptors or enzymes are predictive of the efficacy of these compounds in vivo (Rabbani et al., Int. J. Cancer 63: 840-845, 1995).

Such assays employ transwell tissue culture inserts. Invasive cells are defined as cells which are able to traverse through the Matrigel® and upper aspect of a polycarbonate membrane and adhere to the bottom of the membrane. Transwells (Costar) containing polycarbonate membranes (8.0 μm pore size) are coated with Matrigel® (Collaborative Research), which has been diluted in sterile PBS to a final concentration of 75 μg/mL (60 μL of diluted Matrigel® per insert), and placed in the wells of a 24-well plate. The membranes are dried overnight in a biological safety cabinet, then rehydrated by adding 100 μL of DMEM containing antibiotics for 1 hour on a shaker table. The DMEM is removed from each insert by aspiration and 0.8 mL of DMEM/10% FBS/antibiotics is added to each well of the 24-well plate such that it surrounds the outside of the transwell (“lower chamber”). Fresh DMEM/antibiotics (100 μL), human Glu-plasminogen (5 μg/mL), and any inhibitors to be tested are added to the top, inside of the transwell (“upper chamber”). The cells which are to be tested are trypsinized and resuspended in DMEM/antibiotics, then added to the top chamber of the transwell at a final concentration of 800,000 cells/mL. The final volume of the upper chamber is adjusted to 200 μL. The assembled plate is then incubated in a humid 5% CO₂ atmosphere for 72 hours. After incubation, the cells are fixed and stained using DiffQuik® (Giemsa stain) and the upper chamber is then scraped using a cotton swab to remove the Matrigel® and any cells which did not invade through the membrane. The membranes are detached from the transwell using scalpel blade, mounted on slides using Permount® and cover-slips, then counted under a high-powered (400×) field. An average of the cells invaded is determined from 5-10 fields counted and plotted as a function of inhibitor concentration.

D. Tube-Formation Assays of Anti-Angiogenic Activity

The compounds of this invention are tested for their anti-angiogenic activity in one of two different assay systems in vitro.

ECs, for example, HUVEC or human microvascular ECs (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch).

This assay is recognized in the art to be predictive of angiogenic (or anti-angiogenic) efficacy iii vivo (Min, H Y et al., Cancer Res. 56: 2428-2433,1996).

In an alternate assay, EC tube formation is observed when ECs are cultured on Matrigel® (Schnaper et al., J. Cell. Physiol. 165:107-118 1995). ECs (˜10⁴ cells/well) are transferred onto Matrigel®-coated 24-well plates, and tube formation is quantitated after 48 hrs. Inhibitors are tested by adding them either at the same time as the ECs or at various time points thereafter. Tube formation can also be stimulated by adding (a) angiogenic growth factors such as bFGF or VEGF, (b) differentiation stimulating agents (e.g., PMA) or (c) a combination of these.

This assay models angiogenesis by presenting to the ECs a particular type of basement membrane, namely the layer of matrix which migrating and differentiating ECs might be expected to first encounter. In addition to bound growth factors, the matrix components found in Matrigel® (and in basement membranes in situ) or proteolytic products thereof may also be stimulatory for EC tube formation which makes this model complementary to the fibrin gel angiogenesis model previously described (Blood et al., Biochim. Biophys. Acta 1032:89-118, 1990; Odedra et al., Pharmac. Ther. 49:111-124, 1991). The MEK-inhibitory compounds of this invention will inhibit EC tube formation in both assays, reflective of their anti-angiogenic capability.

E. Assays for the Inhibition of EC or Tumor Cell Proliferation

The ability of the compounds of the invention to inhibit the proliferation of EC's may be determined in a 96-well format. Type I collagen (gelatin) is used to coat the wells of the plate (0.1-1 mg/mL in PBS, 0.1 n per well for 30 minutes at room temperature). After washing the plate (3× w/PBS), 3-6,000 cells are plated per well and allowed to attach for 4 hrs (37° C./5% CO₂) in Endothelial Growth Medium (EGM; Clonetics) or M199 media containing 0.1-2% FBS. The media and any unattached cells are removed at the end of 4 hrs and fresh media containing bFGF (1-10 ng/mL) or VEGF (1-10 ng/mL) is added to each well. Compounds to be tested are added last and the plate is allowed to incubate (37° C./5% CO₂) for 24-48 hrs. MTS (Promega) is added to each well and allowed to incubate from 1-4 hrs. The absorbance at 490 nm, which is proportional to the cell number, is then measured to determine the differences in proliferation between control wells and those containing test compounds.

A similar assay system can be set up with cultured adherent tumor cells. However, collagen may be omitted in this format. Tumor cells (e.g., 3,000-10,000/well) are plated and allowed to attach overnight. Serum free medium is then added to the wells, and the cells are synchronized for 24 hrs. Medium containing 10% FBS is then added to each well to stimulate proliferation. Compounds to be tested are included in some of the wells. After 24 hrs, MTS is added to the plate and the assay developed and read as described above.

F. Phenotypic Conversion and Cell Growth Inhibition Assays

1. Soft Agar Growth or Colony Formation in Suspension

Normal cells require a solid substrate to attach and grow. Oncogenic transformation results in loss of this phenotype so that the cells grow detached from the substrate. For example, transformed cells can grow in stirred suspension culture or suspended in semisolid medium, such as semisolid or soft agar. Techniques for soft agar growth or colony formation in suspension assays are described in Freshney, supra and Garkavtsev, I. et al., Nat Genet 14:415-20 (1996).

Transformed cells successfully treated with LF or a proteolytic derivative or homologue reverts to the normal phenotype-anchorage dependence. Thus, assays measuring growth or colony formation in soft agar can be used to identify LF homologues, mimetics, and modulators. This type of assay will identify LF genetic constructs that, when expressed in a transformed cell, reverse the phenotype.

2. Growth Limitation by Contact inhibition and Cell Density

Normal adherent typically grow in a flat, organized pattern in culture until they contact neighboring cells. Such contact causes growth to cease, a phenomenon termed “contact inhibition.” Transformed, however, do not obey these rules and are impervious to contact inhibition; hence they continue to grow to high densities in disorganized foci. This is evident morphologically as a disoriented monolayer of cells or rounded cells in foci within the regular pattern of normal surrounding cells. Labeling with [³H]-thymidine at saturation density serves as one measure of growth and its limitation by cell density. See Freshney, supra.

LF will cause transformed cells to revert to the normal phenotype and show contact inhibition and cessation of growth cultures at a lower cell density.

Assays measuring contact inhibition and density limitation of growth can be used to identify LF constructs, derivatives, etc. which are inhibit abnormal proliferation of transformed host cells. Transformed cells, for example of a long-term cell line are used. Delivery of LF to these cells, either as protein or via DNA expression, would reinstate contact inhibition and lower saturation densities.

In one embodiment, the labeling index with [³H]-thymidine at saturation density is used to measure density limitation on growth. Transformed host cells are provided with LF in a form that is expressed intracellularly, and are grown for 24 hours at saturation density in a non-limiting culture medium. The percentage of cells labeling with [³H]-thymidine can be is determined autoradiographically. See, Freshney, supra. Expression of LF would result in a lower labeling index compared to control transformed cells (e.g., transfected with a vector lacking an insert).

3. Dependence on Growth Factors or Serum

Growth factor or serum dependence can be used as a basis for assaying for functional LF constructs or other MEK inhibitors. Transformed cells have a lower serum dependence than their normal counterparts (Temin, H, J. Natl. Cancer Inst. 37:167-175 (1966); Eagle, H. et al., J. Exp. Med. 131:863-879 (1970)); Freshney, supra). This is in part due to release of autocrine growth factors by the transformed cells. When an LF protein is present in a transformed cell (such as by expression of a transfected gene) the cell would release lower amounts of growth factors and would become serum dependent Therefore, measuring this parameter is useful for identifying LF constructs which could function as antiangiogenic cancer therapeutics.

F. Assays of Cytotoxicity

The anti-proliferative and cytotoxic effects of the compositions may be determined for various cell types including tumor cells, ECs, etc. Anti-proliferative effects would be expected against tumor cells and stimulated ECs but, under some circumstances not quiescent ECs or normal human dermal fibroblasts.

A typical assay would involve plating cells at a density of 5-10×10³ cells/well in a 96-well plate. The compound to be tested is added at varying concentrations and allowed to incubate with the cells for 30 minutes. The cells are washed 3× with media, then fresh media containing ³H-thymidine (e.g., 1 μCi/mL) is added to the cells and they are allowed to incubate at 37° C. in 5% CO₂ for 24 and 48 hours. Cells are lysed at the various time points using 1 M NaOH and counts per well determined using a β-counter. Proliferation may be measured non-radioactively using MTS reagent or CyQuant® to measure total cell number. For cytotoxicity assays (measuring cell lysis), a Promega 96-well cytotoxicity kit is used. If there is evidence of anti-proliferative activity, induction of apoptosis may be measured using TumorTACS® (Genzyme).

G. Caspase-3 Activity

The ability of the compounds of the invention to promote apoptosis of EC's may be determined by measuring activation of caspase-3. Type I collagen (gelatin) is used to coat a P100 plate and 5×10⁵ ECs are seeded in EGMF containing 10% FBS. After 24 hours (at 37° C. in 5% CO₂) the medium is replaced by EGM containing 2% FBS, 10 ng/ml bFGF and the desired test compound. The cells are harvested after 6 hours, cell lysates prepared in 1% Triton and assayed using the EnzChek® Caspase-3 Assay Kit #1 (Molecular Probes) according to the manufactures' instructions.

In Vivo Study of LF in Angiogenesis

A. Corneal Angiogenesis Model

The protocol used is essentially identical to that described by Volpert et al. (J Clin. Invest. 98:671-679 (1996)). Briefly, female Fischer rats (120-140 gms) are anesthetized and pellets (5 □1) comprised of Hydron®, bFGF (150 nM), and the compounds to be tested are implanted into tiny incisions made in the cornea 1.0-1.5 ml from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

B. Matrigel® Plug Assay

This assay may be performed essentially as described by Passaniti et al. (Lab Invest. 67:519-528 (1992)). Matrigel® is maintained at 4° C. until use. Just prior to injection, Matrigel® is mixed with an angiogenic composition (e.g., 100 ng/mL bFGF, 100 ng/mL VEGF), then injected s.c. into mice (0.5 mL per mouse). The injected Matrigel® forms a palpable solid gel which persists for 10 days, at which time the animals are euthanized. The Matrigel® plugs are removed and angiogenesis quantitated by measuring the amount of hemoglobin in the Matrigel® plugs or by counting neovessels in sections prepared from the plugs. Anti-CD31 staining may be used to confirm neovessel formation and microvessel density in the plugs. (CD-31 is also known as platelet-endothelial cell adhesion molecule.)

C. Chick Chorioallantoic Membrane (CAM) Angiogenesis Assay

This assay is performed essentially as described by Nguyen et al. (Microvascular Res. 47:31-40 (1994)). A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

D. In Vivo Assessment Angiogenesis Inhibition and Anti-Tumor Effects Using the Matrigel® Plug Assay with Tumor Cells

In this assay, tumor cells, for example 1-5×10⁶ cells of the 3LL Lewis lung carcinoma or the rat prostate cell line MatLyLu, are mixed with Matrigel® and then injected into the flank of a mouse following the protocol described in Sec. B., above. A mass of tumor cells and a powerful angiogenic response can be observed in the plugs after about 5 to 7 days. The anti-tumor and anti-angiogenic action of a compound in an actual tumor environment can be evaluated by including it in the plug. Measurement is then made of tumor weight, Hb levels or fluorescence levels (of a dextran-fluorophore conjugate injected prior to sacrifice). To measure Hb or fluorescence, the plugs are first homogenize with a tissue homogenizer.

E. Xenogaft Model of Subcutaneous (s.c.) Tumor Growth

Nude mice are inoculated with tumor cells and Matrigel® (1×10⁶ cells in 0.2 mL) s.c. in the right flank of the animals. In addition to the tumor exemplified herein, other tumors well-studied in the art may be used, e.g., MDA-MB-231 cells (human breast carcinoma).

The tumors are staged to 200 mm³ and then treatment with a test composition is initiated (100 μg/animal/day given q.d. IP). Tumor volumes are obtained every other day and the animals are sacrificed after 2 weeks of treatment. The tumors are excised, weighed and paraffin embedded. Histological sections of the tumors are analyzed by H and E, anti-CD31, Ki-67, TUNEL, and CD68 staining.

F. Xenograft Model of Metastasis

LF or other antitumor agents of this invention are also tested for inhibition of late metastasis using an experimental metastasis model (Crowley, C W et al., Proc. Natl. Acad. Sci. USA 90 5021-5025 (1993)). Late metastasis involves the steps of attachment and extravasation of tumor cells, local invasion, seeding, proliferation and angiogenesis. Human prostatic carcinoma cells (PC-3) transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This permits utilization of either of these markers (fluorescence detection of GFP or histochemical calorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human prostate cancer. For example, GFP-expressing PC-3 cells (1×10⁶ cells per mouse) are injected iv into the tail veins of nude (nu/nu) mice. Animals are treated with a test composition at 100□g/animal/day given q.d. IP. Single metastatic cells and foci are visualized and quantitated by fluorescence microscopy or light microscopic histochemistry or by grinding the tissue and quantitative colorimetric assay of the detectable label.

G. Models for Testing Inhibition of Spontaneous Metastasis In Vivo by LF and Functional Homologues

The rat syngeneic breast cancer system (Xing et al., Int. J. Cancer 67:423-429 (1996) employs Mat BIII rat breast cancer cells. Tumor cells, e.g., 10⁶ in 0.1 mL PBS, are inoculated into the mammary fat pads of 10 female Fisher rats. At the time of inoculation, a 14-day Alza osmotic mini-pump is implanted intraperitoneally to dispense the polypeptide. The polypeptide is dissolved in PBS (200 mM stock), sterile filtered and placed in the minipump to achieve a dispensing rate of about 4 mg/kg/day. Control animals receive vehicle (PBS) alone or a control polypeptide in the minipump. Animals are euthanized at day 14.

In the rats treated with the MEK inhibitors of this invention, there is a significant reduction in the size of the primary tumor and in the number of metastases in the spleen, lungs, liver, kidney and lymph nodes (enumerated as discrete foci). Upon histological and immunohistochemical analysis, it is seen that in treated animals, there is increased necrosis and signs of apoptosis. Large necrotic areas are seen in tumor regions lacking in neovascularization. In contrast, treatment with control polypeptides fail to cause a significant change in tumor size or metastasis.

H. 3LL Lewis Lund Carcinoma: Primary Tumor Growth

This tumor line arose spontaneously in 1951 as carcinoma of the lung in a C57BL/6 mouse (Cancer Res 15:39, 1955. See, also Malave, I. et al., J. Nat'l. Canc. Inst. 62:83-88 (1979)). It is propogated by passage in C57BL/6 mice by subcutaneous (sc) inoculation and is tested in semiallogeneic C57BL/6×DBA/2 F₁ mice or in allogeneic C3H mice. Typically six animals per group for subcutaneously (sc) implant, or ten for intramuscular (im) implant are used. Tumor may be implanted sc as a 2-4 mm fragment, or im or sc as an inoculum of suspended cells of about 0.5-2×10⁶-cells. Treatment begins 24 hours after implant or is delayed until a tumor of specified size (usually approximately 400 mg) can be palpated. The test compound is administered ip daily for 11 days

Animals are followed by weighing, palpation, and measurement of tumor size. Typical tumor weight in untreated control recipients on day 12 after inoculation is 500-2500 mg. Typical median survival time is 18-28 days. A positive control compound, for example cyclophosphamide at 20 mg/kg/injection per day on days 1-11 is used. Results computed include mean animal weight, tumor size, tumor weight, survival time For confirmed therapeutic activity, the test composition should be tested in two multi-dose assays.

I. 3LL Lewis Lung Carcinoma: Primary Growth and Metastasis Model

This model has been utilized by a number of investigators. See, for example, Gorelik, E. et al., J. Nat'l. Canc. Inst. 65:1257-1264 (1980); Gorelik, E. et al., Rec. Results Canc. Res. 75:20-28 (1980); Isakov, N. et al., Invasion Metas. 2:12-32 (1982); Talmadge J E et al., J. Nat'l. Canc. Inst. 69:975-980 (1982); Hilgard, P. et al., Br. J. Cancer 35:78-86(1977)). Test mice are male C57BL/6 mice, 2-3 months old. Following sc, im, or intra-footpad implantation, this tumor produces metastases, preferentially in the lungs. With some lines of the tumor, the primary tumor exerts anti-metastatic effects and must first be excised before study of the metastatic phase (see also U.S. Pat. No. 5,639,725).

Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected subcutaneously, either in the dorsal region or into one hind foot pad of C57BL/6 mice. Visible tumors appear after 3-4 days after dorsal sc injection of 10⁶ cells. The day of tumor appearance and the diameters of established tumors are measured by caliper every two days.

The treatment is given as one or two doses of polypeptide, per week. In another embodiment, the polypeptide is delivered by osmotic minipump.

In experiments involving tumor excision of dorsal tumors, when tumors reach about 1500 mm³ in size, mice are randomized into two groups: (1) primary tumor is completely excised; or (2) sham surgery is performed and the tumor is left intact. Although tumors from 500-3000 mm³ inhibit growth of metastases, 1500 mm³ is the largest size primary tumor that can be safely resected with high survival and without local regrowth. After 21 days, all mice are sacrificed and autopsied.

Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ¹²⁵IdUrd into lung cells (Thakur, M L et al., J. Lab. Clin. Med. 89:217-228 (1977). Ten days following tumor amputation, 25 μg of fluorodeoxyuridine is inoculated into the peritoneums of tumor-bearing (and, if used, tumor-resected mice). After 30 min, mice are given 1 μCi of ¹²⁵IdUrd (iododeoxyuridine). One day later, lungs and spleens are removed and weighed, and a degree of ¹²⁵IdUrd incorporation is measured using a gamma counter.

In mice with footpad tumors, when tumors reach about 8-10 mm in diameter, mice are randomized into two groups: (1) legs with tumors are amputated after ligation above the knee joints, or (2) mice are left intact as nonamputated tumor-bearing controls. (Amputation of a tumor-free leg in a tumor-bearing mouse has no known effect on subsequent metastasis, ruling out possible effects of anesthesia, stress or surgery). Mice are killed 10-14 days after amputation. Metastases are evaluated as described above.

Statistics: Values representing the incidence of metastases and their growth in the lungs of tumor-bearing mice are not normally distributed. Therefore, non-parametric statistics such as the Mann-Whitney U-Test may be used for analysis.

Study of this model by Gorelik et al. (1980, supra) showed that the size of the tumor cell inoculum determined the extent of metastatic growth. The rate of metastasis in the lungs of operated mice was different from primary tumor-bearing mice. Thus in the lungs of mice in which the primary tumor had been induced by inoculation of larger doses of 3LL cells (1-5×10⁶) followed by surgical removal, the number of metastases was lower than that in nonoperated tumor-bearing mice, though the volume of metastases was higher than in the nonoperated controls. Using ¹²⁵IdUrd incorporation as a measure of lung metastasis, no significant differences were found between the lungs of tumor-excised mice and tumor-bearing mice originally inoculated with 1×10⁶ 3LL cells. Amputation of tumors produced following inoculation of 1×10⁵ tumor cells dramatically accelerated metastatic growth. These results were in accord with the survival of mice after excision of local tumors. The phenomenon of acceleration of metastatic growth following excision of local tumors had been repeatedly observed (for example, see U.S. Pat. No. 5,639,725). These observations have implications for the prognosis of patients who undergo cancer surgery.

For a compound to be useful in accordance with this invention, it should demonstrate activity in at least one of the above (in vitro or in vivo) assay systems.

Pharmaceutical and Therapeutic Compositions and Their Administration

The compounds that may be employed in the pharmaceutical compositions of the invention include all of those compounds described above, as well as the pharmaceutically acceptable salts of these compounds. Pharmaceutically acceptable acid addition salts of the compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts.

Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine.

As stated above, the compounds of the invention possess the ability to inhibit the MAPK pathway and to inhibit angiogenesis, properties that are exploited in the treatment of cancer, in particular metastatic cancer. A composition of this invention may be active per se, or may act as a “pro-drug” that is converted in vivo to the active form.

The compounds of the invention, as well as the pharmaceutically acceptable salts thereof, may be incorporated into convenient dosage forms, such as capsules, impregnated wafers, tablets or injectable preparations. Solid or liquid pharmaceutically acceptable carriers may be employed. Preferably, the compounds of the invention are administered systemically, e.g., by injection or infusion. When used, injection may be by any known route, preferably intravenous, subcutaneous, intramuscular, intracranial or intraperitoneal. Infusion is preferably by the intravenous route. Injectables or infusible preparations can be prepared in conventional forms, either as solutions or suspensions, solid forms suitable for solution or suspension in liquid prior to injection or infusion, or as emulsions.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington 's Pharmaceutical Sciences, Mack Publishing Company, Easton Pa. (Gennaro 18th ed. 1990).

The pharmaceutical preparations are made following conventional techniques of pharmaceutical chemistry, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the treatment of any of a number of animal genera and species, and are equally applicable in the practice of human or veterinary medicine. Thus, the pharmaceutical compositions can be used to treat domestic and commercial animals, including birds and more preferably mammals, as well as humans.

Though the preferred routes of administration are conventional systemic routes. The term “systemic administration” refers to administration of a composition or agent such as the polypeptide, peptides or small organic molecule herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial (or lung instillation), intracranial, intra-aural or intraocular. The term “local administration” refers to administration of a composition or drug into a limited, or circumscribed, anatomic space, such as intratumoral injection into a tumor mass or peritumoral in the vicinity of a tumor, as well as subcutaneous (s.c.) and intramuscular (i.m.) injection. One of skill in the art would understand that local administration or regional administration often also result in entry of a composition into the circulatory system, i.e., so that s.c. or i.m. are also routes for systemic administration. Injectables or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, e.g., as an ointment, cream or gel; orally; rectally; e.g., as a suppository.

For topical application, the compound may be incorporated into topically applied vehicles such as a salve or ointment. The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Preferred vehicles for non-sprayable topical preparations include ointment bases, e.g., polyethylene glycol-1000 (PEG-1000); conventional creams such as HEB cream; gels; as well as petroleum jelly and the like.

Also suitable for topic application are sprayable aerosol preparations wherein the compound, preferably in combination with a solid or liquid inert carrier material, is packaged in a squeeze bottle or in admixture with a pressurized volatile, normally gaseous propellant. The aerosol preparations can contain solvents, buffers, surfactants, perfumes, and/or antioxidants in addition to the compounds of the invention.

For the preferred topical applications, especially for humans, it is preferred to administer an effective amount of the compound to an affected area, e.g., skin surface, mucous membrane, eyes, etc. This amount will generally range from about 0.001 mg to about 1 g per application, depending upon the area to be treated, the severity of the symptoms, and the nature of the topical vehicle employed.

Antiangiogenic compositions may be administered in combination with a biodegradable, biocompatible polymeric implant which releases the troponin active agent over a controlled period of time at a selected site. Examples of preferred polymeric materials include polyanhydrides, polyorthoesters, polyglycolic acid, polylactic acid, polyethylene vinyl acetate, and copolymers and blends thereof. See, for example, Medical Applications of Controlled Release, Langer and Wise (eds.), 1974, CRC Press, Boca Raton, Fla.; Controlled Drug Bioavailability, Drug Product Design and Performance, Smolen and Ball (eds.), 1984, Wiley, N Y; Ranger et al., 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105. In another embodiment, a controlled release system can be placed in proximity of the therapeutic target, e.g., the brain, thus requiring only a fraction of the systemic dose (Goodson, In: Medical Applications of Controlled Release, supra, vol. 2, pp. 115-138). Other controlled release systems are discussed in a review by Langer, R, 1990, Science 249:1527-1533)

Other pharmaceutically acceptable carriers for the present compositions, particularly polypeptides, are liposomes, pharmaceutical compositions in which the active protein is contained either dispersed or variously present in corpuscles consisting of aqueous concentric layers adherent to lipidic layers. The active polypeptide or peptide, or the nucleic acid is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Therapeutic compositions for treating tumors and cancer may comprise, in addition to the LF or homologue, one or more additional anti-tumor agents, such as mitotic inhibitors, e.g., vinblastine; alkylating agents, e.g., cyclophosphamide; folate inhibitors, e.g., methotrexate, piritrexim or trimetrexate; antimetabolites, e.g., 5-fluorouracil and cytosine arabinoside; intercalating antibiotics, e.g. adriamycin and bleomycin; enzymes or enzyme inhibitors, e.g., asparaginase, topoisomerase inhibitors such as etoposide; or biological response modifiers, e.g., interferons or interleukins. In fact, pharmaceutical compositions comprising any known cancer therapeutic in combination with LF as disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

Therapeutic Methods

The methods of this invention may be used to inhibit tumor growth and invasion in a subject or to suppress angiogenesis induced by tumors. By inhibiting the growth or invasion of a tumor or angiogenesis, the methods result in inhibition of tumor metastasis. A vertebrate subject, preferably a mammal, more preferably a human, is administered an amount of the compound effective to inhibit tumor growth, invasion or angiogenesis. The compound or pharmaceutically acceptable salt thereof is preferably administered in the form of a pharmaceutical composition as described above.

Doses of the compounds preferably include pharmaceutical dosage units comprising an effective amount of the polypeptide. By an effective amount is meant an amount sufficient to achieve a steady state concentration in vivo which results in a measurable reduction in any relevant parameter of disease and may include growth of primary or metastatic tumor or a measurable prolongation of disease-free interval or of survival. For example, a reduction in tumor growth in 20% of patients is considered efficacious (Frei III, E., The Cancer Journal 3:127-136 (1997)). However, an effect of this magnitude is not considered to be a minimal requirement for the dose to be effective in accordance with this invention.

In one embodiment, an effective dose is preferably 10-fold and more preferably 100-fold higher than the 50% effective dose (ED₅₀) of the compound in an in vivo assay as described herein.

The amount of active compound to be administered depends on the precise polypeptide or derivative selected, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

A preferred dose for treating a subject, preferably mammalian, more preferably human, with a tumor is an amount of up to about 100 milligrams of active compound per kilogram of body weight. A typical single dosage of the polypeptide is between about 1 ng and about 100 mg/kg body weight. For topical administration, dosages in the range of about 0.01-20% concentration (by weight) of the compound, preferably 1-5%, are suggested. A total daily dosage in the range of about 0.1 milligrams to about 10 grams is preferred for intravenous administration. The foregoing ranges are, however, suggestive, as the number of variables in an individual treatment regime is large, and considerable excursions from these preferred values are expected.

To some degree, the combination of dose and route of the active antiangiogenic composition is selected based on known toxicities. Thus, an intratumoral or peritumoral route of administration is preferred over i.v. administration when the effective dose has toxic side effects when given systemically but less toxic or no toxic side effects when given into, or in the vicinity of, the tumor.

An effective amount or dose of the polypeptide for inhibiting cellular or enzymatic activity in vitro is in the range of about 1 picogram to about 10 nanograms per cell. Effective doses and optimal dose ranges may be determined in vitro using the methods described herein.

The compounds of the invention may be characterized as producing an inhibitory effect on endothelial cell or tumor cell proliferation, migration, invasion, or on angiogenesis or on tumor metastasis. The compounds are especially useful in producing an anti-tumor effect in a mammalian host, preferably human, harboring a tumor.

Diseases and Disorders to be Treated

The foregoing compositions and treatment methods are useful for inhibiting cell proliferation or angiogenesis in a subject having any disease or condition associated with undesired cell proliferation, angiogenesis or metastasis. Malignant and metastatic diseases and conditions (tumors and cancer) which can be treated in accordance with the present invention include, but are not limited to, solid tumors, e.g., carcinomas, sarcomas, lymphomas and other malignant or nonmalignant tumors such as those listed in the table below (for a review of such disorders, see any textbook of clinical oncology, most recent edition, e.g. Cancer: Principles & Practice of Oncology, 5^{th} Ed. (DeVita, V. et al., eds), Philadelphia: Lippincott-Raven Publishers, 1997)

Cancers/Tumors

Acoustic neuroma
Adenocarcinoma
Angiosarcoma
Astrocytoma
Basal cell carcinoma
Bile duct carcinoma
Bladder carcinoma
Breast cancer
Bronchogenic carcinoma
Cervical cancer
Chondrosarcoma
Choriocarcinoma
Colon carcinoma
Craniopharyngioma
Cystadenocarcinoma
Embryonal carcinoma
Endotheliosarcoma
Ependymoma
Ewing's tumor
fibrosarcoma
glioma
hemangioblastoma
hepatoma
Kaposi's sarcoma
leiomyosarcoma
liposarcoma
lung carcinoma
lymphangiosarcoma
lymphangioendotheliosarcoma
medullary carcinoma
medulloblastoma
melanoma
meningioma
mesothelioma
myxosarcoma
neuroblastoma
oligodendroglioma
osteogenic sarcoma
ovarian cancer
pancreatic cancer
papillary adenocarcinomas
pinealoma
prostate cancer
renal cell carcinoma
retinoblastoma
rhabdomyosarcoma
sebaceous gland carcinoma
seminoma
small cell lung carcinoma
squamous cell carcinoma
sweat gland carcinoma
synovioma
testicular tumor
Wilms' tumor

Other classes of diseases associated with undesired or uncontrolled angiogenesis that may be treated in accordance with this invention include atherosclerosis, myocardial angiogenesis, angiofibroma, arteriovenous malformations, post-balloon angioplasty vascular restenosis, neointima formation following vascular trauma, vascular graft restenosis, coronary collateral formation, deep venous thrombosis, and ischemic limb angiogenesis.

Ocular neovascularization is a leading cause of blindness in the world (Lee et al., Surv. Ophthalmol. 43:245-269 (1998)). The most common diseases caused by this process are proliferative diabetic retinopathy, neovascular age-related macular degeneration, and retinopathy of prematurity (Neely, K A et al., Am. J. Path. 153:665-670 (1998)). The present pharmaceutical compositions are intended for the treatment of any of the above or other diseases or conditions that involve ocular neovascularization, chief among them, sickle cell retinopathy, retinal vein occlusion, neovascular glaucoma, retrolental fibroplasia, uveitis, diseases associated with choroidal or iris neovascularization, corneal graft neovascularization, as well as other eye inflammatory diseases or ocular tumors.

Other disorders which can be treated in accordance with the present invention include, but are not limited to, uterine disease such as endometriosis, hemangioma, arthritis, psoriasis, delayed wound healing, granulations, hemophilic joints, hypertrophic scars, nonunion fractures, Osler-Weber syndrome, pyogenic granuloma, scleroderma, trachoma, and vascular adhesions. An important class of conditions treatable by the present antiangiogenic compositions are fibrosis associated with chronic inflammatory conditions. This includes some of the conditions listed above as well as hemophilic joints, hypertrophic scars, telangiectasia, pyogenic granuloma, Von-Hippel-Landau syndrome, trachoma, vascular adhesions, lung fibrosis, chemotherapy-induced fibrosis, wound healing with scarring and fibrosis, peptic ulcers, fractures, keloids. Also included are disorders of vasculogenesis, hematopoiesis, ovulation, menstruation, pregnancy and placentation, or any other disease or condition in which invasion or angiogenesis is pathogenic or undesired.

Certain brain tumors are among the most highly neovascularized tumors known. The present pharmaceutical compositions are therefore intended for the treatment of any of a number of brain tumors, including but not limited to glioblastoma; glioblastoma multiformae; various astrocytomas such as anaplastic astrocytoma, pilocytic astrocytoma, pleomorphic xanthoastrocytoma, subependymal giant cell astrocytoma, fibrillary astrocytoma, gemistocytic astrocytoma, protoplasmic astrocytoma; mixed oligoastrocytoma and malignant oligoastrocytoma; oligodendroglioma and anaplastic oligodendroglioma; ependymoma, including anaplastic ependymoma, myxopapillary ependymoma, and subependymoma;

Endometriosis (mentioned above) is a condition in which ectopic endometrium is present in abnormal locations, the ovary being the most common site. Adenomyosis is a similar condition in which endometrial tissue has penetrated the uterine myometrium. Endometriotic tissue resembles neoplastic tissue in its ability to implant and invade. Accordingly, the present pharmaceutical compositions are intended for the treatment of endometriosis, adenomyosis, endometrial carcinoma and endometrioid tumors of the ovary

More recently, it has become apparent that angiogenesis inhibitors may play a role in preventing inflammatory angiogenesis and gliosis following traumatic spinal cord injury, thereby promoting the reestablishment of neuronal connectivity (Wamil, A W et al., 1998, Proc. Nat 'l. Acad. Sci. USA 95:13188-13193). Therefore, the MEK-inhibitor compositions of the present invention are administered as soon as possible after traumatic spinal cord injury and for several days up to about two weeks thereafter to inhibit the angiogenesis and gliosis that would physically prevent reestablishment of neuronal connectivity. The treatment reduces the area of damage at the site of spinal cord injury and facilitates regeneration of neuronal function and thereby prevents paralysis. The compounds of the invention are expected also to protect axons from Wallerian degeneration, and reverse aminobutyrate-mediated depolarization (occurring in traumatized neurons).

Therapeutic or prophylactic utility of the present invention and the determination of therapeutically effective dosages can be determined or demonstrated in vivo in a suitable animal model system prior to testing in humans. Such model systems may be based on the use of rats, mice, chicken, cows, monkeys, rabbits, etc. For in vivo testing, prior to administration to humans, any animal model system known in the art may be used. Some preferred model systems have been set forth above.

Having now generally described the invention, the same will be more readily understood through reference to the following examples which are provided by way of illustration, and are not intended to be limiting of the present invention, unless specified.

### EXAMPLE I

### Methods

Cell Lines and Treatments

NIH3T3 (490) cells (mouse fibroblasts) expressing either the empty vector (pDCR) or transforming human H-ras V12) protein (Webb, C. P et al. (1998) Proc. Natl. Acad. Sci. USA 95, 8773-8778) were grown in DMEM supplemented with 10% fetal bovine serum, 1% penicillin/streptomycin and maintained at 37° C. in a humidified atmosphere of 5% CO₂. PA, inactive LF(E687C), and LF were purified from cultures of B. anthracis as described elsewhere (Leppla, S H (1988) in Meth Enzymol, ed., Harshman, S. (Academic Press, Inc., Orlando) pp. 103-116).

Cell Morphology, Immunoblotting and Immunostaining

Cells were cultured in 10 cm dishes or on slides under the conditions described above. When cells reached 30-50% confluence, one of the following agents was added to the culture medium:

(a) 100 ng/ml PA plus inactive 10 ng/ml LF(E687C) (Klimpel, K R et al. (1994) Mol Microbiol 13, 1093-1100),

(b) PA plus LF (100 ng/ml PA plus 10 ng/ml LF), or

(c) PD98059 (50 μM from a 50 mM stock in DMSO).

Cells were cultured a further 24 h., at which point cells were lysed for immunoblotting (10 μg protein/lane) or fixed for immunostaining as outlined previously (Duesbery, N S et al. (1997) Proc Natl Acad Sci USA 94, 9165-70; Fukasawa, K et al. (1997) Mol. Cell Biol. 17, 506-518) using antibodies specific for one of the following proteins:

1. human MEK1 (NT, 1:1000; Upstate Biotechnology, Lake Placid, N.Y.),

2. phosphorylated MAPK (pTEpY, 1:4000; Promega, Madison, Wis.),

3. MAPK1/2 (K-23/C-14, 1:4000; Santa Cruz Biotechnology, Santa Cruz, Calif.),

4. cathepsin L (M−19, 1:100; Santa Cruz Biotechnology),

5. β-tubulin (TUB2.1, 1:1000; Sigma, St. Louis, Mo.) or

6. actin (AC-40, 1:250; Sigma)

along with Oregon Green-conjugated anti-mouse antibody (1:250; Molecular Probes, Eugene Oreg.). Slides were examined by confocal laser scanning microscopy.

Cell Proliferation Analysis

Cells were cultured in 96-well plates under the conditions described above.

When cells reached 30-50% confluence, one of the following agents was added to the culture medium:

(a) 100 ng/ml PA plus inactive 10 ng/ml LF(E687C),

(b) PA plus LF (100 ng/ml PA plus 10 or 1 ng/ml LF (hi and lo respectively)), or

(c) PD98059 (25 μM from a 50 mM stock in DMSO).

Cells were cultured a further 48 h. at which point cell proliferation was assayed using the CellTiter 96® (Aqueous Non-Radioactive Cell Proliferation Assay (Promega) according to the manufacturer's instructions. Results are D presented relative to proliferation of control samples incubated in the presence of culture medium alone and as a mean of at least three measurements±standard deviation.

In Vitro Tumorigenicity Assays

Soft Agar Colony Formation

Trypsinized cells were washed with Ca²⁺/Mg²⁺-free PBS, resuspended at a concentration of 1×10⁴ cells/ml in DMEM containing 10% calf serum, 0.5% (w/v) Noble agar (Difco laboratories, MI), in the presence or absence of LeTx (100 ng/ml PA plus 10 ng/ml LF) as indicated below, and layered over a 0.5 ml solid plug of DMEM containing 1% agar in 24 well plates. Cells were incubated at 37° C., 5% CO₂, for one week during which the cells were monitored daily. The images shown in FIG. 3 were made at the end of this time. Each sample was assayed in triplicate in three separate experiments.

Extracellular Matrix Invasion Assay

Three-dimensional Matrigel (Becton Dickinson) invasion assays were performed as described in the art (Jeffers, M et al.(1996) Mol Cell Biol 16, 1115-25). Approximately 2.5×10⁴ cells were mixed with growth factor-reduced Matrigel® supplemented with

1. medium alone,

2. inactive LeTx (100 ng/ml PA plus 10 ng/ml LF(E687C)) or

3. LeTx (100 ng/ml PA plus 10 ng/ml LF).

The cell suspension was cultured for up to one week during which the cells were monitored daily. The images shown in FIG. 3 were made at day 4. Each sample was assayed in triplicate in three separate experiments.

In Vivo Tumorigenicity Assays

V12H-ras transformed NIH 3T3 cells (10⁵ cells in a volume of 100 μl) were injected subcutaneously into 10 athymic nude mice on the left and right sides of the back behind the last rib. When tumors reached a size of 5-7 mm (approximately 2 weeks), the mice were divided into two groups as follows:

Group A: sham-injected (insertion of the needle only) intra-tumorally in the left side and injected in the right side with 100 μl buffered saline.

Group B: injected in the left side with 100 μl buffered saline and in the right side with 10 μg PA plus 2 μg LF in 100 μl buffered saline.

Injections continued once daily for a total period of five days. The sizes of the tumors were monitored following injection. When control tumors attained a diameter of 20 mm (approximately 3-4 weeks) the mice were euthanized and the tumors dissected, trimmed, and fixed in formalin for further analyses. Paraffin tissue sections (5 μm) were stained using mouse monoclonal antibodies against one of two endothelial cell markers, CD31 (BBa7, 1:100; Research Diagnostics, Flanders, N.J.) and CD 34 (M7 165, 1:25; DAKO, Carpinteria, Calif.), followed by FITC conjugated secondary antibodies. Slides were imaged using a Zeiss CLSM 410 confocal microscope. Images were made of representative sections that showed histologic features of a fibrosarcoma using a water immersion 40× high numerical aperture lens and were stored digitally.

### EXAMPLE II

### Cellular Consequences of LeTx Treatment

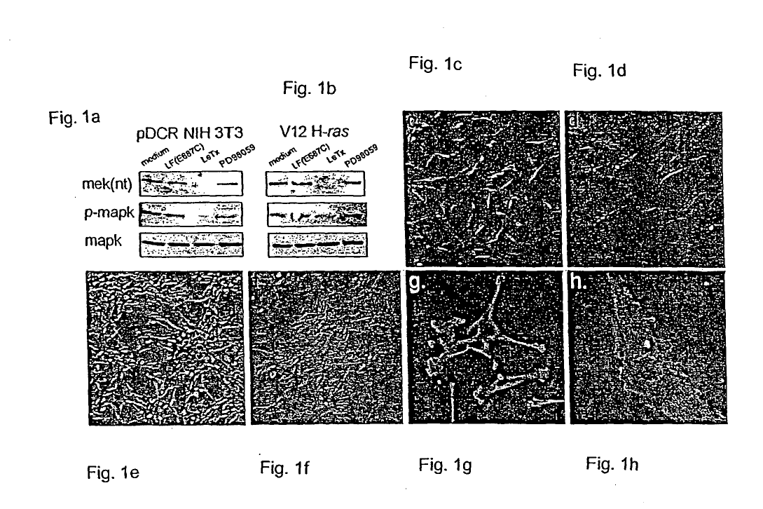

To test the effects of LeTx upon NIH 3T3 cells we first confirmed that LeTx was active upon both non-transformed and oncogenic V12H-ras-transformed NIH 3T3 cells by performing immunoblot analyses upon lysates of cells which had been incubated 24 h. in the presence of inactive LeTx (PA plus LF(E687C) which had a point mutation in its Zn²⁺-binding site), LeTx, or PD98059, a small organic compound that preferentially inhibits MEK1 activation (Alessi, D R et al. (1995) J. Biol. Chem. 270, 27489-27494; Dudley et al., supra. (FIGS. 1A and 1B). As previously by the present inventors and colleagues, treatment of both V12H-ras-transformed and non-transformed NIH 3T3 cells with LeTx, but not inactive LeTx or PD98059, caused the loss of NH₂-terminal epitopes of MEK1, indicating that LF had cleaved intracellular MEK1.

We assayed the consequences of proteolysis on downstream MAPK activity using antibodies specific for phosphorylated (active) MAPK. While the levels of total MAPK remained constant under all conditions in both cell types, levels of phosphorylated MAPK decreased in response to LeTx, but not inactive LeTx. Treatment of cells with PD98059 caused a similar but less pronounced reduction in levels of phosphorylated MAPK in each cell type.

It is well established that NIH 3T3 cells transformed by oncogenic V12 H-ras undergo a change in morphology from a irregular, flattened shape to a spindle-like form with an associated loss in actin stress fibers (Dudley et al., supra) FIGS. 1C, 1E). LeTx treatment was accompanied by a reversion of the shape of V12H-ras transformed NIH 3T3 cells from a spindle-like shape (FIGS. 1E, 1G) to an irregular, flattened shape (FIG. 1f) with an enlarged nucleus and prominent actin stress-fibers (FIG. 1H). Treatment of cells with PA plus inactive LF(E687C) had no effect on cell morphology. Similarly, it has been reported that treatment of cells with PD98059 (or U0126, a compound with an action PD98059 but that inhibits both MEK1 and MEK2 (Favata, MF et al. (1998) J Biol Chem 273, 18623-32), causes morphological reversion of transformed cells (Dudley et al., supra; Fukazawa, H et al. (2000) Cancer Res 60, 2104-7).

MEK/MAPK signaling is known to play an important role in mitogenesis. To determine the effects of LeTx upon cell growth, we cultured cells in 96-well plates in the presence or absence of PA plus LF or inactive LF(E687C) for 48 hours (Table I). For comparison, we also tested the effects of PD98059.

TABLE I
The effects of LeTx upon cell proliferation

| | | PA + LF | | | 25 μM | 50 μM |
|---|---|---|---|---|---|---|
| Cell type | untreated | (E687C) | lo LeTx | hi LeTx | PD98059 | PD98059 |
| NIH 3T3 | 1 | 1.00 ± 0.04 | 0.81 ± 0.08 | 0.63 ± 0.07 | 0.70 ± 0.09 | 0.47 ± 0.03 |
| V12 H-ras | 1 | 0.94 ± 0.02 | 1.05 ± 0.02 | 0.87 ± 0.11 | 0.79 ± 0.07 | 0.77 ± 0.03 |
| Cell proliferation was assayed in non-transformed (pDCR) or V12 H-ras-transformed NIH 3T3 cells treated with either medium alone, 100 ng/ml PA plus inactive 10 ng/ml LF(E687C), PA plus LF (100 ng/ml PA plus 10 or 1 ng/ml LF (hi and lo respectively)), or PD98059 (25 μM or 50 μM from a 50 mM stock in DMSO) as described in the methods section. Data is presented relative to proliferation of control samples incubated in the presence of | | | | | | |
| # culture medium alone and as an average of at least three measurements plus and minus standard deviation about the mean. | | | | | | |

Treatment with 50 μM PD98059 for 48 hrs. caused a 50% inhibition of growth of non-transformed NIH 3T3 cells, whereas only a 25% inhibition was found in cells transformed with V12H-ras. In contrast, treatment with LeTx caused only a modest (20-35%) inhibition of non-transformed NIH 3T3 cells proliferation and did not significantly inhibit proliferation of V12H-ras-transformed cells.

### EXAMPLE II

### Effects of LeTx on Anchorage Independent Growth and Invasion

Tumor growth and invasion is a complex multistep process that involves anchorage independent growth, motility, and proteolytic degradation of the extracellular matrix. Aspects of these processes may be simulated in vitro by measuring a cell's ability to (i) grow independent of substrate adhesion and form colonies in a soft agar suspension, and (ii) degrade an artificial extracellular matrix (basement membrane Matrigel®).

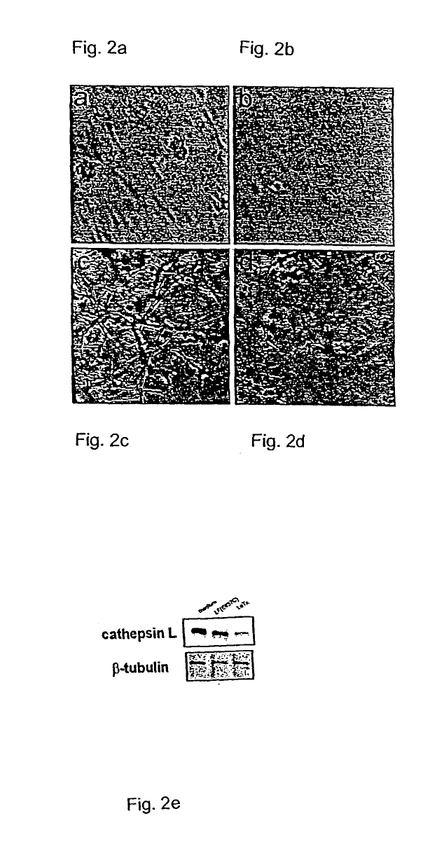

Non-transformed NIH 3T3 cells suspended in soft agar fail to proliferate and remain as single cells in suspension (Yang, J J et al. (1998) Mol Cell Biol 18, 2586-95). By contrast, V12H-ras-transformed cells continue to proliferate in the absence of substrate adhesion and form colonies of cells. However, LeTx completely prevented colony formation of V12 H-ras-transformed cells (FIGS. 2a, b). Moreover, LeTx prevented V12H-ras-transformed cells from invading, or branching into, the extracellular matrix (FIGS. 2c, 2d). Invasiveness in Matrigel has been correlated with the expression of extracellular matrix-degradative enzymes such as the MAPK-dependent cysteine protease, cathepsin L (Janulis, M et al., (1999) J Biol Chem 274, 801-13; Silberman, S et al. (1997) J Biol Chem 272, 5927-35).

To determine whether LeTx inhibited cathepsin L expression, we treated lysates of cells with LeTx and subjected them to immunoblot analysis with antibodies to cathepsin L. Our results clearly demonstrated that LeTx blocked cathepsin L expression in V12H-ras-transformed cells (FIG. 2e).

### EXAMPLE IV

### Effects of LeTx on In Vivo Tumorigenicity

Because various in vitro assays do not always mimic adequately all aspects of tumorigenicity or always predict therapeutic value, we assessed the effects of LeTx in vivo. We d V12 H-ras-transformed cells were injected subcutaneously into the left and right dorsal areas of 10 athymic nu/nu mice.

Preliminary experiments suggested that intratumoral injections of LeTx also had systemic effects on growth of the distal tumors. Therefore we divided the mice into two groups, A and B, when tumors had grown to a diameter of 5-7 mm.

Group A tumors were sham-injected on the left side and injected on the right side with buffered saline. Group B tumors were injected on the left side with LeTx (10 μg PA and 2 μg LF) and on the right side with buffered saline. We continued these injections once daily for a total of five days and monitored tumor size (length×width) thereafter. Tumors in mice of Group A were considerably larger than those in Group B (FIG. 3). We did not observe differences between the left and right side tumors of mice within each same group. Importantly, several tumors from mice in group B regressed in size over the course of the experiment. Moreover, while it was apparent that the average mass of a Group B tumor (1.35±0.77 g) was significantly lower than that of control Group A tumors (3.67±1.25 g) (Student's t-test, p=0.002), there were no significant differences in the masses of left and right side tumors of mice within the same group.

These results also showed that LeTx injected into one Group B tumor could systemically affect growth of the contralateral tumor of the same animal. Remarkably, using this dose regimen all LeTx injected mice appeared healthy in all respects throughout the course of the experiment.

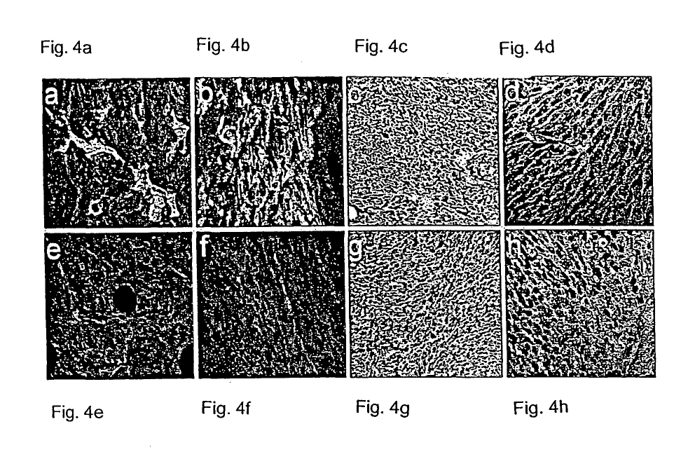

Histological examination of tumors excised from both groups of mice revealed classic fibrosarcomas with high mitotic indices (FIGS. 4c, 4g). While all tumors examined showed some degree of non-inflammatory necrosis, tumors derived from the LeTx-treated Group B mice showed more necrosis than did tumors from control Group A mice. In addition, the external coloration of tumors removed from the two groups differed markedly: group A tumors were mottled red-purple, while those from group B were uniformly pale yellow (FIGS. 4c, 4d). This indicated that LeTx inhibited tumor angiogenesis in vivo.

To verify the anti-angiogenic effect, we immunostained sections of these tumors with antibodies to CD31 (FIGS. 4a, 4e) and CD34 (FIGS. 4b, 4f), markers of vascularization (Webb, C et al. (2000) J Neurooncology 50:71-87), and found that levels of each marker were dramatically reduced in tumors taken from LeTx-injected mice.

### EXAMPLE IV

### Discussion of Examples I-IV

MEK inhibitors such as PD98059, U0126, and PD184352 inhibit tumor cell growth in vitro or in vivo (Dudley et al., supra; Favata et al., supra; Sebolt-Leopold et al., supra. Each differs somewhat in its substrate specificity and affinity. PD98059 inhibits MEK1, and to a lesser extent MEK2, with IC₅₀'s of ≅4 and 50 μM, respectively (Alessi et al., supra). U0126 inhibits both MEK1 and MEK2 with IC₅₀'s of ≅0.07 and 0.06 μM, respectively (Favata et al., supra). U0126 was shown to inhibit p70^{S6K} phosphorylation through a MEK-independent mechanism. PD184352 inhibits MEK1 with an IC₅₀ of ≅0.02 μM (Sebolt-Leopold et al., supra).

LF has been demonstrated to inactivate MEK1 but because it cleaves MEK2 (Duesbery et al., 1998, supra; Vitale, G et al. (1999) J Appl Microbiol 87, 288) and MEK3 (Pellizzari et al., supra), it is believed to inactivate these kinases as well. Furthermore, based on sequence homology it is likely that additional LF substrates will be found within the MEK family (Duesbery et al., 1999, supra) or other regulatory transduction pathways. Indeed, the present inventors and colleagues have found that LF also cleaves MEK4, MEK6 and MEK7 in vitro.

Treatment of V 12H-ras-transformed cells with LeTx caused rapid and dramatic alteration of cell morphology and the appearance of actin-stress fibers. This effect is likely to be mediated by inhibition of MEK1 and/or MEK2 activity because similar effects are induced by PD98059 and U0126 (Fukuzawa et al., supra).

Treatment of V12H-ras-transformed cells with LeTx was also shown to inhibit soft agar colony formation as well as extracellular matrix invasion in vitro. Further, we found that LeTx inhibited expression of cathepsin L, an enzyme involved in degradation of the extracellular matrix whose expression requires high levels of MAPK activity in ras-transformed NIH 3T3 cells (Janulis et al., Silberman et al., supra). Again, because (i) similar changes in soft agar colony formation have been reported to occur following treatment with U0126 and (ii) the expression of dominant negative MAPK1 and MAPK2 inhibits the ability of V12 H-ras-transformed cells to grow in soft agar and to invade Matrigel basement membrane, it is concluded that these changes are mediated primarily by the LeTx inhibition of MEK1 and/or MEK2.

LeTx had only a modest effect on cell proliferation when compared to PD98059 (Dudley et al., supra) or PD184352 (Sebolt-Leopold et al., supra) whereas LeTx was a more effective inhibitor of MAPK than was PD98059 in V12 H-ras transformed cells (see FIGS. 1a, 1b). Thus, the activities of LF and the small molecule MEK inhibitors are distinct.

Intravenous (vs. intratumoral) injection of a similar dose of LeTx might have been expected to have undesired side effects (given that the LD₅₀ in mice is 12.5 μg PA and 2.5 μg LF (Ezzell, J W et al. (1984) Infect. Immun. 45:761-767; Welkos, S L et al. (1986) Infect. Immun. 51:795-800)). However, because the toxin was injected intratumorally, the amount of toxin which became blood-borne and capable of systemic spread may have been considerably lower by this route of administration.

Each of the in vitro assays discussed above measures a particular aspect or aspects of the transformed cell phenotype. Although useful, these assays provide less valuable information than does evaluation of actual tumor growth in vivo. For example, the rate at which a primary tumor develops in vivo is not simply a reflection of the rate at which its cells divide but rather is a function of a complex series of cellular activities that include proliferation, vascularization, and invasion.

Our results clearly demonstrate that LeTx reduces tumor growth in vivo. That this was achieved in part by cell death is evident from the necrotic appearance of the tumors from LeTx injected animals. The cause of this cell death is not entirely clear since in vitro studies failed to demonstrate cytotoxic effects. Possibly sustained LeTx treatment of cells would eventually have led to cytotoxicity in vitro. Alternatively, the tumor necrosis may have resulted from poor vascularization that we indeed observed in tumors in LeTx injected animals.

The mechanism by which LeTx inhibited vascularization was not revealed by these studies. Although a role for MEK/ERK activity in angiogenic growth factor signaling has been reported (Gupta, K et al. (1999) Exp Cell Res 247, 495-504; Redlitz, A et al. (1999) J Vasc Res 36, 28-34; Yu, Y et al. (1999) J Cell Physiol 178, 235-46; Rousseau, S et al. (1997) Oncogene 15, 2169-77; Anand-Apte, B et al. (1997) J Biol Chem 272, 30688-92; Eliceiri, B P et al. (1998) J Cell Biol 140, 1255-63), it is important to note that Sebolt-Leopold et al. (supra) did not disclose any effects of PD184352 on tumor vascularization. Expression of dominant negative MEK in murine angiosarcoma cells inhibited growth in soft agar but had no effect on tumorigenicity of xenografts in nude mice (LaMontagne Jr, K R et al. (2000) Am J Pathol 2000 157, 1937-45). However, the induction of fibroblast growth factor (FGF)-binding protein, which binds and stabilizes the angiogenic stimulator FGF (Wu, DQ et al. (1991) J Biol Chem 266, 16778-16785; Czubayko, F et al. (1994) J Biol Chem 269, 28243-28248),⁴ has recently been shown to depend upon both MEK/MAPK and p38 signal transduction pathways (Harris, V K et al. (2000) J Biol Chem 275, 10802-11). Thus, it is concluded that the combined inhibition of these pathways by LeTx is results in effective inhibition of tumor vascularization and concomitant growth.

In conclusion, these results show that LeTx has therapeutic activity that exceeds what is expected based solely upon its MEK1 inhibitory activity. Use of LeTx or LF in vivo is a novel strategy for inhibiting and reversing tumor growth and therefore, for treating cancer in a subject.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A method for inhibiting cell migration, cell invasion, cell proliferation or angiogenesis, or for inducing apoptosis, comprising contacting cells associated with undesired cell migration, invasion, proliferation or angiogenesis with an effective amount of an inhibitor of MEK or of an enzyme that is a member of the MAPK family.

2. A method for inhibiting angiogenesis comprising contacting cells associated with undesired angiogenesis with an effective amount of an inhibitor of MEK or of an enzyme that is a member of the MAPK family.

3. The method of claim 1 or 2, wherein said inhibitor of MEK is a MEK-directed protease.

4. The method of claim 3, wherein said protease is Bacillus anthracis lethal factor or a functional derivative thereof.

5. The method of claim 1 or 2 wherein said inhibitor is an organic small molecule.

6. The method of claim 5 wherein said inhibitor is PD98059, U0126 or PD184352.

7. The method of claim 1 or 2 wherein the inhibitor is an inhibitor of a MAPK family member selected from the group consisting of ERK 1, ERK2, p38 kinase and JNK.

8. The method of claim 7 wherein the MAPK family member is p38 kinase.

9. The method of claim 8 wherein the inhibitor is SB203580.

10. The method of any of claims 1-9, wherein said contacting is in vivo.

11. The method of claim 10 wherein said contacting is in a mammalian subject that has a tumor and said inhibition of angiogenesis results in cessation of growth or a measurable regression of a primary or metastatic tumor.

12. The method of claims 10 or 11 wherein said in vivo contacting is performed in a human.

13. A method for inhibiting angiogenesis in a mammalian subject, comprising administering to a mammalian subject in need of such inhibition an angiogenesis-inhibiting amount of a pharmaceutical composition that comprises:
(a) an inhibitor of MEK or of an enzyme that is a member of the MAPK family; and
(b) a pharmaceutically acceptable carrier or excipient, thereby inhibiting said angiogenesis.

14. A method for treating a mammalian subject having a disease or condition associated with undesired cell migration, invasion, proliferation, or angiogenesis, comprising administering to the subject an effective amount of a pharmaceutical composition that comprises:
(a) an inhibitor of MEK or of an enzyme that is a member of the MAPK family; and
(b) a pharmaceutically acceptable carrier or excipient, thereby treating said subject.

15. A method for treating a mammalian subject having a disease or condition associated with undesired angiogenesis or neovascularization, comprising administering to the subject an effective amount of a pharmaceutical composition that comprises:
(a) an inhibitor of MEK or of an enzyme that is a member of the MAPK family; and
(b) a pharmaceutically acceptable carrier or excipient, thereby treating said subject.

16. The method of claim 13, 14 or 15, wherein said inhibitor of MEK is a MEK-directed protease.

17. The method of claim 16, wherein said protease is Bacillus anthracis lethal factor or a functional derivative thereof.

18. The method of claim 13, 14 or 15 wherein said inhibitor is an organic small molecule.

19. The method of claim 18 wherein said inhibitor is PD98059, U0126 or PD184352.

20. The method of claim 13, 14 or 15 wherein the inhibitor is an inhibitor of a MAPK family member selected from the group consisting of ERK 1, ERK2, p38 kinase and JNK.

21. The method of claim 7 wherein the MAPK family member is p38 kinase.

22. The method of claim 8 wherein the inhibitor is SB203580.

23. The method of any of claims 13-22 wherein said subject is a human.

24. The method of any of claims 14-23 wherein said disease or condition is tumor growth, tumor invasion or tumor metastasis.

25. The method of any of claim 13-23 wherein said subject has a tumor, and said angiogenesis inhibition results in reduction in size or growth rate of said tumor or destruction of said tumor.

26. The method of claim 24 or 25 wherein said tumor is a solid tumor.

27. The method of claim 26 wherein said tumor is a brain tumor.

28. A method according to any of claims 14-23 wherein said disease or condition is atherosclerosis, myocardial angiogenesis, angiofibroma, arteriovenous malformation, post-balloon angioplasty vascular restenosis, vascular adhesions, neointima formation following vascular trauma, vascular graft restenosis, coronary collateral formation, deep venous thrombosis, lung fibrosis, chemotherapy-induced fibrosis, wound healing with scarring and fibrosis, hypertrophic scar, endometriosis, uterine adenomyosis, hemangioma, arthritis, psoriasis, pyogenic granuloma, delayed wound healing, a nonunion fracture, Osler-Weber syndrome, scleroderma, trachoma, fibrosis associated with chronic inflammatory conditions, telangiectasia, Von-Hippel-Landau syndrome, peptic ulcer or keloids.

29. A method according to any of claims 14-23 wherein said disease or condition is an ocular disease selected from proliferative diabetic retinopathy, neovascular age-related macular degeneration, retinopathy of prematurity, sickle cell retinopathy, retinal vein occlusion, neovascular glaucoma, retrolental fibroplasia, uveitis, choroidal neovascularization, iris neovascularization or corneal graft neovascularization.
